(19) United States
(12) Patent Application Publication
(10) Pub. No.: US 2020/0027536 A1
(43) Pub. Date: Jan. 23, 2020

(54) INFORMATION PROCESSING SYSTEM AND INFORMATION PROCESSING APPARATUS

(71) Applicant: FUJITSU LIMITED, Kawasaki-shi (JP)

(72) Inventors: Hideyuki Arai, Numadu (JP); Hiroyuki Katayama, Numazu (JP); Akito YAMAZAKI, Kawasaki (JP); Yuri Shimada, Yokohama (JP)

(73) Assignee: FUJITSU LIMITED, Kawasaki-shi (JP)

(21) Appl. No.: 16/504,719

(22) Filed: Jul. 8, 2019

(30) Foreign Application Priority Data
Jul. 17, 2018 (JP) 2018-134181

(51) Int. Cl. G16H 10/60; G16H 50/70; G06F 3/06

(52) U.S. Cl. CPC G16H 10/60; G06F 3/0605; G06F 3/0631; G16H 50/70

(57) ABSTRACT

An information processing apparatus includes: a processor configured to: allocate, upon receiving referral information of a patient from a first medical institution, a physical volume from among a plurality of physical volumes; generate volume information that indicates the physical volume; cause an information processing device to store a copy of the examination information of the patient in the physical volume, the examination information of the patient being stored in a first database; receive the volume information from a second medical institution; identify the physical volume based on the received volume information; allocate the identified physical volume to the information processing device; and cause the information processing device to obtain the copy of the examination information of the patient from the physical volume and store the obtained copy of the examination information in a second database.

### CROSS-REFERENCE TO RELATED APPLICATION

This application is based upon and claims the benefit of priority of the prior Japanese Patent Application No. 2018-134181, filed on Jul. 17, 2018, the entire contents of which are incorporated herein by reference.

### FIELD

The embodiment discussed herein is related to an information processing system and an information processing apparatus.

### BACKGROUND

In recent years, demands for big data analyses are increasing more and more. In order to obtain more accurate and useful analysis results in big data analyses, it is desirable to collect as many data samples as possible.

In order to promote big data analyses in a domestic medical field, Japanese government has a plan to establish a system that collects medical information, such as electronic medical records, and the like from medical institutions all over Japan and utilize the medical information as big data. The medical information collected from the medical institutions all over Japan is the information that is not only utilized for big data analyses but also the information that is very helpful for patients and medical institutions at the time of medical examinations.

Regarding medical information, an information processing apparatus is known that reduces the risk of information leakage at the time of analyzing medical information collected from medical institutions all over Japan. A medical examination information sharing method is also known that guarantees privacy protection of the patient and security when the medical examination data of a patient is shared across a plurality of medical institutions.

A data exchange system is also known that easily and safely exchanges data when data is exchanged among different tenants that are using virtual private clouds of the same cloud computing provider.

Related techniques are disclosed in, for example, Japanese Laid-open Patent Publication No. 2018-28886, Japanese Laid-open Patent Publication No. 10-111897, and Japanese Laid-open Patent Publication No. 2014-200010.

A patient who is receiving treatment at a hospital sometimes changes hospitals for the following reasons:

1. A hospital to which the patient regularly goes does not have sufficient facilities for the treatment.

2. It becomes difficult for the patient to regularly go to the hospital due to relocation, or the like.

3. The patient wants to get a second opinion from another hospital.

When a patient moves to another hospital, it is desirable to transfer medical examination information on illness and injuries currently under treatment to a transfer destination hospital in order to perform the treatment efficiently at the destination hospital. However, the medical examination information is the data that includes much personal information related to the privacy of the patient, and thus there arises a problem with how to ensure security at the time of transferring the medical examination information between the hospitals.

Such a problem occurs not only when medical examination information is transferred between hospitals, but also when medical examination information is transferred among the other medical institutions.

### SUMMARY

According to an aspect of the embodiment, an information processing apparatus includes: a processor configured to: receive referral information of a patient from a first terminal of a first medical institution; allocate, upon receiving the referral information, a predetermined physical volume to be used for storing medical examination information of the patient, from among a plurality of physical volumes included in a storage device; generate volume identification information that indicates the predetermined physical volume; cause an information processing device to store a copy of the medical examination information of the patient in the predetermined physical volume, the medical examination information of the patient being stored in a first database of the first medical institution; receive the volume identification information from a second terminal of a second medical institution; identify the predetermined physical volume based on the volume identification information received from the second terminal; allocate the identified physical volume to the information processing device; and cause the information processing device to obtain the copy of the medical examination information of the patient from the predetermined physical volume and store the obtained copy of the medical examination information in a second database of the second medical institution.

The object and advantages of the invention will be realized and attained by means of the elements and combinations particularly pointed out in the claims.

It is to be understood that both the foregoing general description and the following detailed description are exemplary and explanatory and are not restrictive of the invention.

### BRIEF DESCRIPTION OF DRAWINGS

FIG. 1 is a functional configuration diagram of an information processing system;

FIG. 2 is a flowchart of information processing;

FIGS. 3A and 3B are functional configuration diagrams of a specific example of the information processing system;

FIG. 4 is a diagram illustrating logical volumes of VMs;

FIG. 5 is a diagram illustrating volume corresponding information;

FIG. 6 is a diagram illustrating referral information;

FIG. 7 is a diagram illustrating referral patient information;

FIG. 8 is a diagram illustrating reception information;

FIG. 9 is a diagram illustrating generation processing of integrated analysis target information;

FIGS. 10A, 10B, and 10C are diagrams illustrating a referral sequence;

FIG. 11 is a diagram illustrating a user-ID providing sequence;

FIGS. 12A and 12B are diagrams illustrating a password setting sequence;

FIG. 13 is a diagram illustrating an access code acquisition sequence;

FIGS. 14A, 14B, and 14C are diagrams illustrating a reception sequence;

FIGS. 15A and 15B are diagrams illustrating a reception information generation sequence;

FIGS. 16A and 16B are diagrams illustrating reception information confirmation sequence;

FIGS. 17A, 17B, and 17C are diagrams illustrating a volume allocation sequence;

FIGS. 18A and 18B are diagrams illustrating an information extraction sequence; and

FIG. 19 is a diagram illustrating the hardware configuration of an information processing apparatus.

### DESCRIPTION OF EMBODIMENT

In the following, a detailed description will be given of an embodiment with reference to the drawings.

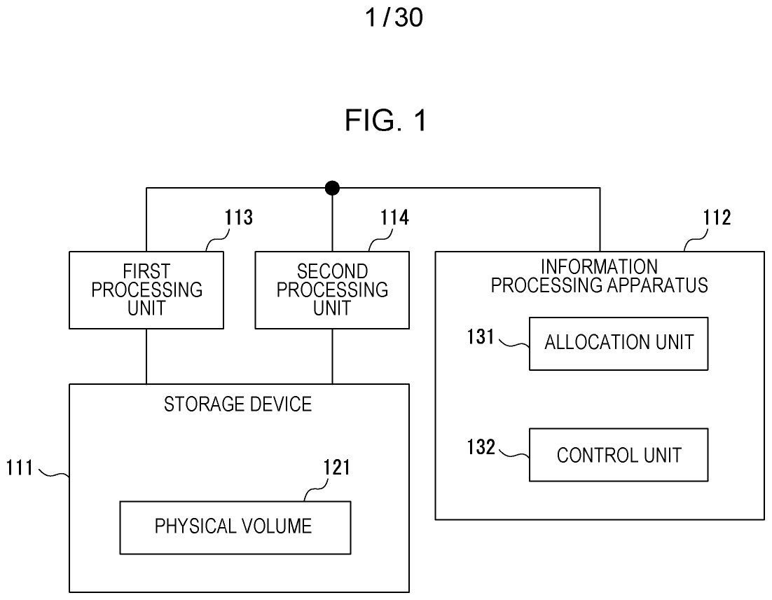

FIG. 1 illustrates an example of the functional configuration of an information processing system according to the embodiment. The information processing system illustrated in FIG. 1 includes a storage device 111, an information processing apparatus (computer) 112, a first processing unit 113, and a second processing unit 114. The storage device 111 includes a physical volume 121, and the information processing apparatus 112 includes an allocation unit 131 and a control unit 132.

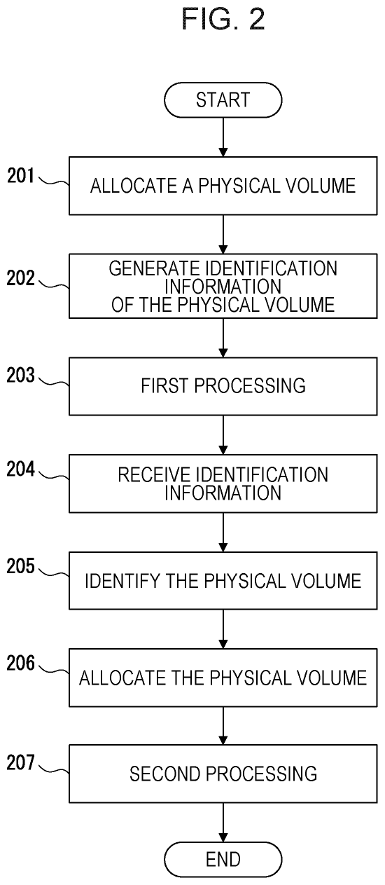

FIG. 2 is a flowchart illustrating an example of information processing performed by the information processing apparatus 112 illustrated in FIG. 1. First, when referral information of a patient of a first medical institution is received, the allocation unit 131 allocates a physical volume 121 in the storage device 111 for a storage area of medical examination information of the patient (step 201). Next, the allocation unit 131 generates identification information indicating the physical volume 121 in the storage device 111, which is given to the patient (step 202).

Next, the control unit 132 causes the first processing unit 113 to perform first processing (step 203). The first processing is processing that stores a copy of the medical examination information of the patient stored in the first database of the first medical institution in the physical volume 121 in the storage device 111.

Next, the allocation unit 131 receives identification information that has been input when the identification information given to the patient is input from a terminal of a second medical institution (step 204). The allocation unit 131 identifies the physical volume 121 in the storage device 111 based on the received identification information (step 205) and allocates the identified physical volume 121 to the second processing unit 114 for a storage area of the medical examination information of the patient (step 206).

Next, the control unit 132 causes the second processing unit 114 to perform second processing (step 207). The second processing is the processing that obtains a copy of the medical examination information from the physical volume 121 in the storage device 111 and stores the copy of the obtained medical examination information in the second database of the second medical institution.

With the information processing system illustrated in FIG. 1, it is possible to safely transfer medical examination information of a patient between medical institutions. For example, the medical institutions are hospitals, clinics, geriatric health services facilities, and the like.

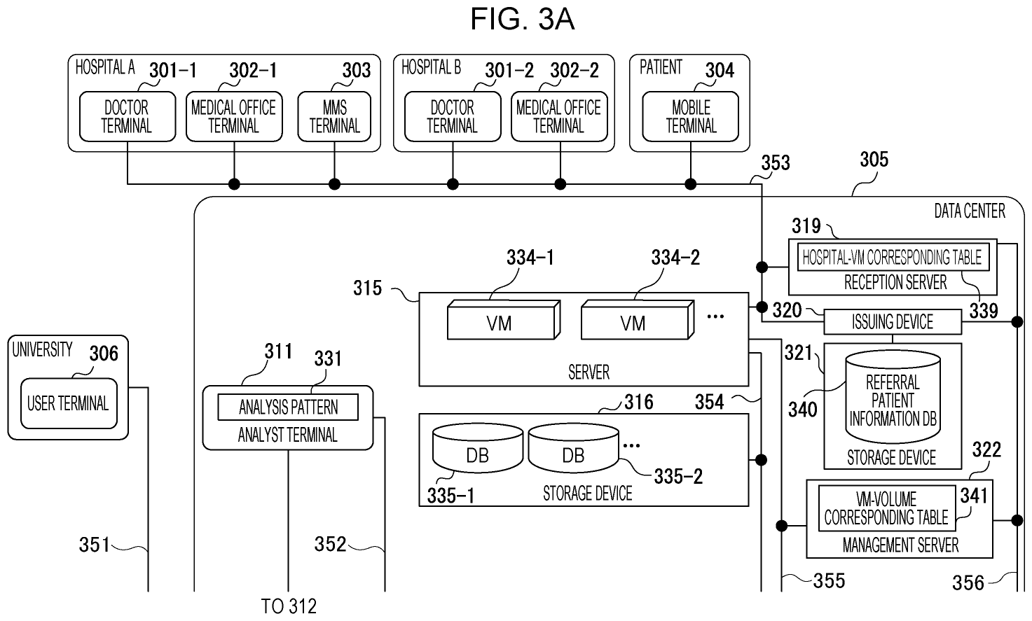

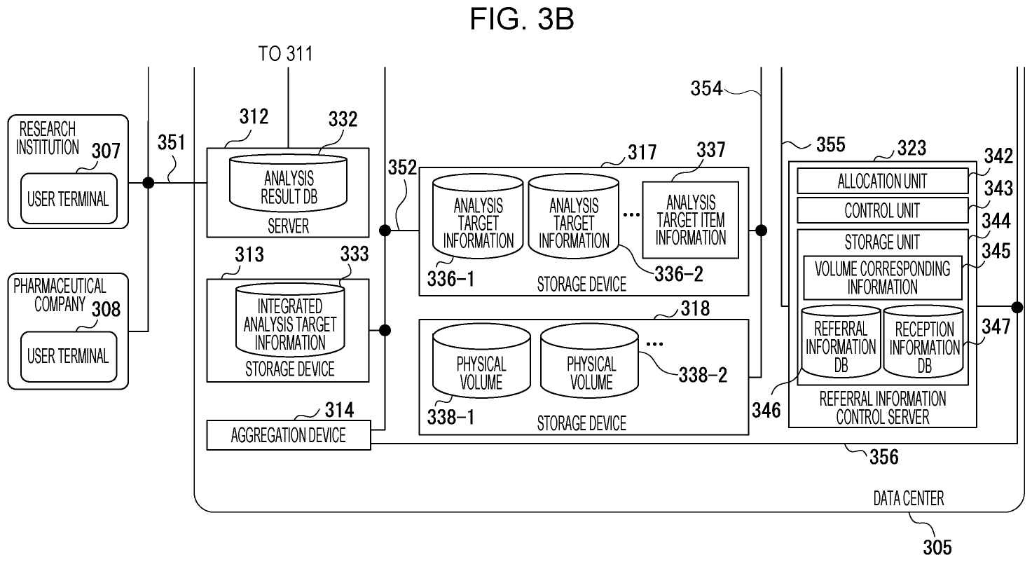

FIGS. 3A and 3B illustrate a specific example of the information processing system illustrated in FIG. 1. The information processing system in FIGS. 3A and 3B includes a doctor terminal 301-1 of a hospital A, a medical office terminal 302-1 of the hospital A, an MMS (multimedia station) terminal 303 of the hospital A, a doctor terminal 301-2 of a hospital B, a medical office terminal 302-2 of the hospital B, and a mobile terminal 304 of a patient. Further, the information processing system includes a data center 305, a user terminal 306 of a university, a user terminal 307 of a research institution, and a user terminal 308 of a pharmaceutical company. The mobile terminal 304 is a smartphone, a tablet, or the like of a patient. The number of hospitals may be three or more.

The data center 305 includes an analyst terminal 311, a server 312, a storage device 313, an aggregation device 314, a server 315, and a storage device 316 to a storage device 318. Further, the data center 305 includes a reception server 319, an issuing device 320, a storage device 321, a management server 322, and a referral information control server 323.

The storage device 318 and the referral information control server 323 correspond to the storage device 111 and the information processing apparatus 112 illustrated in FIG. 1 respectively, and the server 315 corresponds to the first processing unit 113 and the second processing unit 114. The hospital A and the hospital B correspond to the first medical institution and the second medical institution respectively. Hereinafter identification information is sometimes referred to as an ID.

The user terminal 306 to the user terminal 308 and server 312 are connected to a communication network 351, and the analyst terminal 311, the storage device 313, and the aggregation device 314 are connected to a communication network 352. The doctor terminal 301-1, the medical office terminal 302-1, the MMS terminal 303, the doctor terminal 301-2, the medical office terminal 302-2, the mobile terminal 304, the server 315, the reception server 319, and the issuing device 320 are connected to a communication network 353, such as the Internet, or the like.

The server 315 and the storage device 316 to the storage device 318 are connected to a communication network 354, and the server 315, the management server 322, and the referral information control server 323 are connected to the communication network 355. The reception server 319, the issuing device 320, the management server 322, the referral information control server 323, and the aggregation device 314 are connected to a communication network 356.

The server 315 is a cloud server disposed on the communication network 353, and a virtual machine (VM) 334-1 of the hospital A and a VM 334-2 of the hospital B operate in the server 315. For example, when a patient in the hospital A moves to the hospital B, the server 315 operates the VM 334-1 so as to operate as the first processing unit 113 and operates the VM 334-2 so as to operate as the second processing unit 114.

The storage device 316 is a medical examination information storage device, and includes a database (DB) 335-1 of the hospital A and a DB 335-2 of the hospital B. The DB 335-1 stores the medical examination information of patients in the hospital A, and the DB 335-2 stores the medical examination information of patients in the hospital B.

The storage device 317 is an analysis target information storage device and stores analysis target information 336-1 of the hospital A, analysis target information 336-2 of the hospital B, and analysis target item information 337. The analysis target item information 337 is information that specifies an analysis target item among a plurality of items included in the medical examination information of each patient of each hospital. As an analysis target item, for example, an item with which an individual is not identified (i.e., an item other than the personal information of a patient) is specified.

The storage device 318 includes a plurality of physical volumes, such as a physical volume 338-1, a physical volume 338-2, and the like and serves a function of a storage pool. These physical volumes are used for transferring medical examination information between hospitals. One physical volume is allocated to one patient, and medical examination information of a plurality of patients will not be stored in one physical volume.

The reception server 319 stores a hospital-VM corresponding table 339. The hospital-VM corresponding table 339 indicates the association relationship between the hospital A and the hospital B, and the VM 334-1 and the VM 334-2.

When a VM 334-i (i=1, 2) of each hospital is generated, the management server 322 gives a medical institution ID that uniquely identifies the hospital to the VM 334-i. The management server 322 stores a VM-volume corresponding table 341 and allocates physical volumes of a DB 335-i and analysis target information 336-i, and a physical volume 338-j (j is an integer of 1 or more) to each VM 334-i. The VM-volume corresponding table 341 indicates the association relationship between the VM 334-i and the physical volume of the analysis target information 336-i.

The storage device 321 includes a referral patient information DB 340. The referral patient information DB 340 stores referral patient information that includes a user-ID of a patient who moves to another hospital and a volume-ID. The volume-ID is identification information that indicates a physical volume 338-j in the storage device 318. The issuing device 320 gives a volume-ID to the patient while referring to the referral patient information DB 340.

The referral information control server 323 includes an allocation unit 342, a control unit 343, and a storage unit 344 and performs information processing for a referral service. The storage unit 344 stores volume corresponding information 345 and includes referral information DB 346 and reception information DB 347. The allocation unit 342 and the control unit 343 correspond to the allocation unit 131 and the control unit 132 in FIG. 1 respectively.

The volume corresponding information 345 indicates the association relationship between the volume-ID indicating a physical volume 338-j and the location information of the physical volume 338-j. The referral information DB 346 stores referral information including information for taking over the treatment from a former doctor to a succeeding doctor. The reception information DB 347 stores reception information including a preparation state of receiving a patient in a transfer destination hospital.

When referral information of a patient in a hospital A is received, the allocation unit 342 allocates a physical volume 338-j in the storage device 318 for the storage area of the medical examination information of the patient while referring to the volume corresponding information 345. The allocation unit 342 generates a volume-ID indicating the allocated physical volume 338-j. The control unit 343 requests the VM 334-1 in the server 315 to perform processing for transferring the medical examination information of the patient from the hospital A to the hospital B.

The VM 334-1 stores a copy of the medical examination information of the patient in the DB 335-1 in the allocated physical volume 338-j in response to the request from the control unit 343.

When a volume-ID given to the patient is input from the medical office terminal 302-2 of the hospital B, the allocation unit 342 receives the input volume-ID. The allocation unit 342 identifies a physical volume 338-j based on the received volume-ID and allocates the physical volume 338-j to the VM 334-2. The control unit 343 requests the VM 334-2 to perform processing for transferring the medical examination information of the patient from the hospital A to the hospital B.

The VM 334-2 obtains the copy of the medical examination information from the allocated physical volume 338-j in response to the request from the control unit 343 and stores the copy of the obtained medical examination information in the DB 335-2.

The aggregation device 314 obtains the hospital-VM corresponding table 339 from the reception server 319 and obtains the VM-volume corresponding table 341 from the management server 322. Next, the aggregation device 314 identifies a physical volume of the analysis target information 336-i of a specific hospital based on the hospital-VM corresponding table 339 and the VM-volume corresponding table 341. The aggregation device 314 obtains the analysis target information 336-i of the hospital from the storage device 317.

The aggregation device 314 merges the analysis target information 336-1 of the hospital A and the analysis target information 336-2 of the hospital B to generate integrated analysis target information 333, and the storage device 313 stores the integrated analysis target information 333. For example, after a patient of the hospital B is received, it is possible for the aggregation device 314 to merge the analysis target information 336-1 and the analysis target information 336-2 to generate the integrated analysis target information 333.

The analyst terminal 311 stores an analysis pattern 331 and performs analysis processing on the integrated analysis target information 333. The analysis pattern 331 is information that specifies the patterns of analysis processing. The server 312 includes an analysis result DB 332 that stores results of the analysis processing.

In the information processing system in FIGS. 3A and 3B, the storage device 318 for use in transferring medical examination information between hospitals is connected to a communication network 354 and is physically separated from the communication network 353. Normally, a storage dedicated operating system (OS) is installed in the storage device 318, and thus compared with a general purpose OS, the risk of leaking medical examination information from the storage device 318 becomes very low.

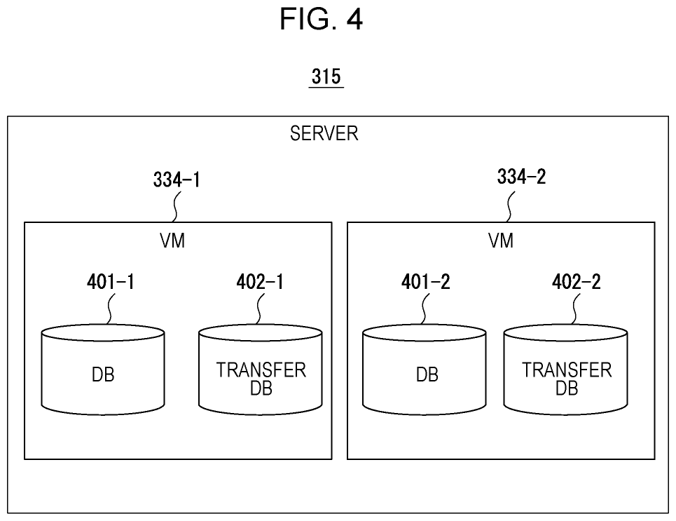

FIG. 4 illustrates an example of logical volumes of the VM 334-i (i =1 and 2) in FIGS. 3A and 3B. The VM 334-i in FIG. 4 includes a DB 401-i and a transfer DB 402-i. The DB 401-i is a logical volume corresponding to the physical volume of the DB 335-i, and the transfer DB 402-i is a logical volume corresponding to the physical volume 338-j. By disposing the VM 334-i for each hospital, the risk of leaking medical examination information between the hospitals becomes low.

When the VM 334-1 generates a copy of the medical examination information, the allocation unit 342 allocates a physical volume 338-j to the transfer DB 402-1. After the completion of copying the medical examination information from the DB 335-1 to the physical volume 338-j, the allocation unit 342 releases the allocation of the physical volume 338-j to the transfer DB 402-1.

Next, when the VM 334-2 obtains the copy of the medical examination information, the allocation unit 342 allocates the physical volume 338-j to the transfer DB 402-2. After the completion of copying the medical examination information from the physical volume 338-j to the DB 335-2, the allocation unit 342 releases the allocation of the physical volume 338-j to the transfer DB 402-2. After that, the medical examination information in the physical volume 338-j is discarded.

Since the medical examination information in the physical volume 338-j is the data that includes much personal information related to the privacy of a patient, it is desirable that only the patient hold the volume-ID of the physical volume 338-j. In this case, the patient presents the volume-ID to the hospital B so that a physical volume of the DB 335-2 is allocated to the transfer DB 402-2 of the VM 334-2, and the VM 334-2 becomes able to refer to the medical examination information.

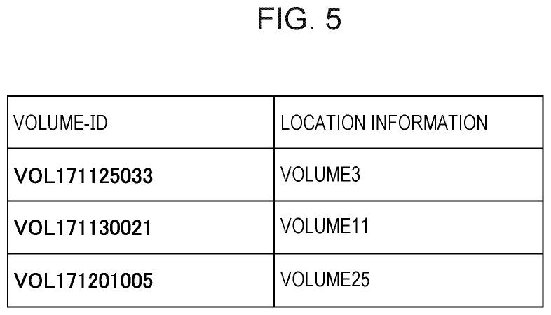

FIG. 5 illustrates an example of the volume corresponding information 345. The volume corresponding information 345 in FIG. 5 includes a volume-ID and location information. A volume-ID is the identification information that indicates the physical volume 338-j, and location information is the location information of the physical volume 338-j. Location information may be the physical address or the logical address of the physical volume 338-j.

It is possible to record the location information of the unused physical volume 338-j in the volume corresponding information 345. When the physical volume 338-j is unused, the corresponding volume-ID field is left blank. When a transfer of medical examination information via a physical volume 338-j is completed, the volume-ID of the physical volume 338-j is deleted.

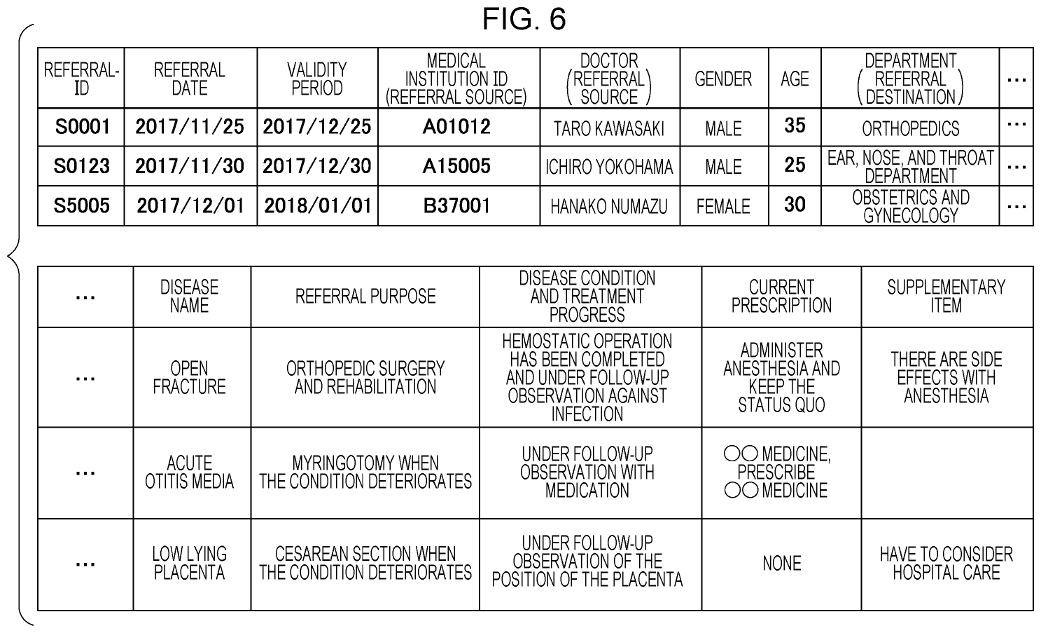

FIG. 6 illustrates an example of the referral information stored in the referral information DB 346. The referral information in FIG. 6 includes a referral-ID, a referral date, a validity period, a medical institution ID (referral source), a doctor (referral source), a gender, an age, a medical department (referral destination), a disease name, a referral purpose, medical conditions and treatment progress, a current prescription, and a supplementary item.

A referral-ID is the identification information that indicates referral information, a referral date represents the date when the referral information was generated, and a validity period is the validity period of the referral information. A medical institution ID (referral source) is the identification information that indicates a hospital where the patient is receiving treatment, and a doctor (referral source) is the name of a doctor who is a referrer. A gender and an age represent the gender and the age of the patient, and a medical department (referral destination) is a medical department suitable for treatment of the patient in a transfer destination hospital.

A disease name represents the name of illness or injury of the patient, a referral purpose represents the purpose of the hospital change, and medical conditions and treatment progress represents an overview of the medical conditions of the patient and the treatment conducted so far. A current prescription represents the prescription currently carried out on the patient, and a supplementary item represents a comment made by the doctor.

At a point in time when referral information is input, a transfer destination hospital is sometimes not determined yet, and thus the referral information in FIG. 6 does not include a name, an address, a date of birth, and the like, which are the personal information of the patient and the information of the transfer destination.

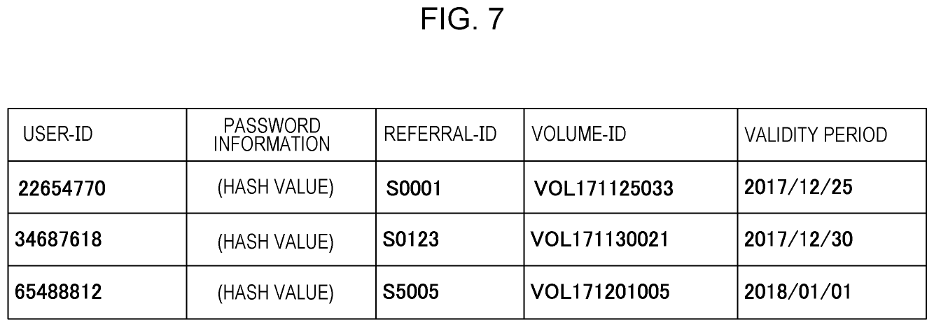

FIG. 7 illustrates an example of the referral patient information stored in the referral patient information DB 340. The referral patient information in FIG. 7 includes a user-ID, password information, a referral-ID, a volume-ID, and a validity period.

A user-ID is identification information given to the patient, and password information represents a hash value of the password set by the patient for the user-ID. A referral-ID and a validity period are the referral-ID and the validity period in FIG. 6 respectively, and a volume-ID is the volume-ID in FIG. 5.

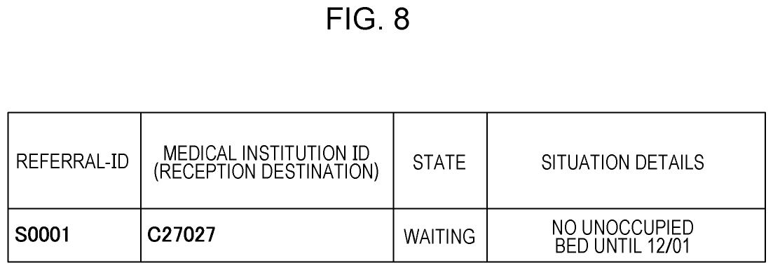

FIG. 8 illustrates an example of the reception information stored in the reception information DB 347. The reception information in FIG. 8 includes a referral-ID, a medical institution ID (reception destination), a state, and situation details. A referral-ID is the referral-ID in FIG. 6, a medical institution-ID (reception destination) is the identification information indicating a transfer destination hospital to receive the patient, a state represents the state of the reception preparations for the patient, and situation details represents the detailed information of the reception preparations for the patient. In this example, for the referral information indicated by the referral-ID “S0001” in FIG. 6, since there are no vacancies of beds until December 1st, a state in which the patient is waiting is recorded. When a vacancy of a bed occurs, the state is changed from waiting to preparation completion.

In the information processing system in FIGS. 3A and 3B, when a transfer destination hospital does not have to make reception preparations for a patient, for example, medical examination information is transferred by the following procedure.

P11. A patient in the hospital A requests a doctor of the hospital A to create referral information to another hospital. The referral information sometimes includes a transfer destination hospital name or sometimes does not include a transfer destination hospital name.

P12. The doctor of the hospital A inputs referral information of the patient in the VM 334-1 using the doctor terminal 301-1, and the VM 334-1 transmits the input referral information to the referral information control server 323.

P13. The referral information control server 323 generates a referral-ID for the patient, adds the referral-ID to the received referral information, and records the referral information in the referral information DB 346.

P14. The referral information control server 323 allocates a physical volume 338-1 to the transfer DB 402-1 of the VM 334-1. The referral information control server 323 generates a volume-ID for the physical volume 338-1 and records the association relationship between the generated volume-ID and the location information of the physical volume 338-1 in the volume corresponding information 345.

P15. The referral information control server 323 issues a user-ID of the patient to the VM 334-1 and notifies the VM 334-1 of the allocation completion of the physical volume 338-1. The issuing device 320 records referral patient information including a user-ID, a referral-ID, and a volume-ID in the referral patient information DB 340.

P16. The VM 334-1 copies the medical examination information of the patient from the DB401-1 to the transfer DB 402-1. When the copy is completed, the referral information control server 323 releases the allocation of the physical volume 338-1 to the transfer DB 402-1.

P17. The VM 334-1 provides the patient with the user-ID via the medical office terminal 302-1 or the MMS terminal 303 installed at the hospital A. In this case, the VM 334-1 may provide the character string of the user-ID or a one-dimensional bar code or a matrix type two-dimensional code that is generated from the user-ID. The VM 334-1 may provide the user-ID in the way of recording in an integrated circuit (IC) card, such as a medical examination card, or the like.

P18. The patient sets a password for the user-ID using the MMS terminal 303. By setting a password for the user-ID, it becomes possible for the patient to login the issuing device 320.

P19. The patient logs in the issuing device 320 using the MMS terminal 303, the mobile terminal 304, or a personal computer (PC) not illustrated in the figure, and obtains an access code including the referral-ID and the volume-ID. The format of the access code may be a character string produced by combining a referral-ID and a volume-ID, or a one-dimensional bar code, or a matrix-type two-dimensional code produced from a referral-ID and a volume-ID.

By managing the association relationship between the volume-ID and the location information of the physical volume 338-1 using the volume corresponding information 345 and providing a patient with a user-ID, it becomes possible only for the patient to obtain the access code to the physical volume 338-1.

P20. The patient presents the access code at the reception of the hospital B, and a clerk of the hospital B inputs the presented access code to the VM 334-2 using the medical office terminal 302-2. The VM 334-2 transmits the input access code to the referral information control server 323.

P21. The referral information control server 323 transmits the access code to the issuing device 320, and the issuing device 320 divides the access code into the referral-ID and the volume-ID and sends them to the referral information control server 323. The referral information control server 323 obtains the location information of the physical volume 338-1 corresponding to the volume-ID from the volume corresponding information 345 and allocates the physical volume 338-1 to the transfer DB 402-2 of the VM 334-2. The referral information control server 323 obtains referral information corresponding to the referral-ID from the referral information DB 346, transmits the referral information to the VM 334-2, and notifies of the allocation completion of the physical volume 338-1.

P22. The VM 334-2 copies the medical examination information of the patient from the transfer DB 402-2 to the DB 401-2. When the copying is completed, the referral information control server 323 releases the allocation of the physical volume 338-1 to the transfer DB 402-2 and discards the medical examination information in the physical volume 338-1. The referral information control server 323 deletes the volume-ID presented by the patient from the volume corresponding information 345.

With the information processing system in FIGS. 3A and 3B, the advantages as follows are obtained.

a. When a patient moves to another hospital, a doctor does not have to work for creating a reference letter in writing, and a patient does not have to present the reference letter to the transfer destination hospital.

b. A reference letter in writing has a limit on the description of medical conditions and treatment progress, an examination result, and the like. It is difficult to provide the transfer destination hospital with all of the medical examination information in process of treatment, and thus re-examination, and the like are sometimes carried out at the transfer destination hospital in order to obtain examination results that are not provided. However, by copying the medical examination information of the original hospital, it becomes possible to provide the transfer destination hospital with all of the medical examination information, and thus re-examination, or the like does not have to be performed.

c. The physical volume 338-j for transferring the medical examination information of a patient between the hospitals is disposed in the independent storage device 318. The VM 334-i of each hospital accesses the physical volume 338-j via the communication network 354 that is separated from the external communication network 353. Accordingly, even an operator of the data center 305 finds it difficult to access the medical examination information in the physical volume 338-j.

d. The patient himself or herself owns the volume-ID of the physical volume 338-j, and the other person does not know the volume-ID. It is possible only for a transfer destination hospital to which the patient presented the volume-ID to refer to the medical examination information of the patient. Accordingly, the possibility of leaking medical examination information to a hospital other than the transfer destination is low, and thus it becomes possible to safely transfer the medical examination information of a patient between the hospitals.

In the information processing system in FIGS. 3A and 3B, when reception preparations for a patient are made at the transfer destination hospital, for example, the following procedure is added between P13 and P14.

P31. The referral information control server 323 records the reception information items to hospital B with the referral-ID in the reception information DB 347.

P32. The referral information control server 323 transmits an input request for inputting the reception information of the patient to the VM 334-2. For example, it is possible for the referral information control server 323 to notify the hospital B of the input request by an email, an RSS, a message notification to the VM 334-2, or a notice when the hospital B has accessed the referral service. RSS refers to Rich Site Summary, RDF Site Summary, or Really Simple Syndication.

P33. A clerk of the hospital B inputs the reception information of the patient in the VM 334-2 using the medical office terminal 302-2, and the VM 334-2 transmits the reception information to the referral information control server 323. The referral information control server 323 adds the referral-ID to the received reception information and records the reception information in the reception information DB 347. When the reception preparations for the patient have not been completed, the clerk regularly updates the reception information until the reception preparations are completed.

P34. When the reception preparations are completed, the referral information control server 323 notifies the VM 334-1 of the hospital A of the completion of the reception preparations. For example, it is possible for the referral information control server 323 to notify the hospital A of the completion of the reception preparations by an email, an RSS, a message notification to the VM 334-1, or a notice when the hospital A has accessed the referral service.

P35. The clerk of the hospital A refers to the reception information of the patient using the medical office terminal 302-1 and confirms the completion of the reception preparations.

When a transfer destination hospital is determined in advance, by providing the referral information control server 323 with the reception information DB 347, it is possible to confirm the state of the reception preparations for the patient at the hospital. Accordingly, it is possible for the original hospital to transfer the medical examination information of the patient after the reception preparations at the transfer destination hospital have been completed, and the transfer destination hospital has become possible to reliably receive the patient.

Incidentally, in an information processing system in Japanese Laid-open Patent Publication No. 2018-28886, there is no consideration in transferring medical examination information to a transfer destination hospital when a patient moves to another hospital. Accordingly, when the medical examination information at each hospital is concealed to generate analysis target information, and the analysis target information of a plurality of hospitals is merged to generate integrated analysis target information, there is a possibility that the medical examination information of the same patient might be collected redundantly from the two places, the original hospital and the transfer destination hospital.

Thus, in the information processing system in FIGS. 3A and 3B, the VM 334-i of each hospital holds a medical institution ID that uniquely identifies a hospital, and medical examination information is identified using the medical institution ID. When integrated analysis target information is generated after a patient of the hospital A moved to the hospital B, for example, analysis target information is collected by the following procedure.

P41. When the VM 334-1 of the hospital A is generated, the management server 322 generates a medical institution-ID indicating the hospital A, and the VM 334-1 holds the medical institution ID.

P42. When the VM 334-2 of the hospital B is generated, the management server 322 generates a medical institution-ID indicating the hospital B, and the VM 334-2 holds the medical institution ID.

P43. A doctor of the hospital A examines a patient and inputs the medical examination information of the patient in the VM 334-1 using the doctor terminal 301-1. The VM 334-1 adds the medical institution ID of the hospital A to the input medical examination information and stores the medical examination information in the DB 335-1 in the storage device 316. The medical examination information of a patient includes a patient name, a date of birth, a gender, an address, a blood type, a health insurance card-ID, a hospital name, an allergy, a prescription, an examination result, a disease name, and the like.

P44. When a patient moves from the hospital A to the hospital B, the medical examination information of the patient in the DB 335-1 is copied to the DB 335-2 in accordance with the procedure of P11 to P22.

P45. After the hospital B received the patient, a doctor of the hospital B examines the patient and inputs the medical examination information of the patient in the VM 334-2 using the doctor terminal 301-2. The VM 334-2 adds the medical institution ID of the hospital B to the input medical examination information and stores the medical examination information in the DB 335-2 of the storage device 316.

P46. The VM 334-1 extracts the medical examination information to which the medical institution ID of the hospital A has been added from the medical examination information in the DB 335-1. The VM 334-1 conceals the extracted medical examination information in accordance with the analysis target item information 337 so as to generate the analysis target information 336-1 and stores the extracted medical examination information in the storage device 317. For example, it is possible for the VM 334-1 to generate the analysis target information 336-1 from the medical examination information using the technique described in Japanese Laid-open Patent Publication No. 2018-28886.

P47. The VM 334-2 extracts the medical examination information to which the medical institution ID of the hospital B is added from the medical examination information in the DB 335-2. The VM 334-2 conceals the extracted medical examination information in accordance with the analysis target item information 337 so as to generate the analysis target information 336-2 and stores the extracted medical examination information in the storage device 317. For example, it is possible for the VM 334-2 to generate the analysis target information 336-2 from the medical examination information using the technique described in Japanese Laid-open Patent Publication No. 2018-28886.

P48. The aggregation device 314 merges the analysis target information 336-1 and the analysis target information 336-2 to generate integrated analysis target information 333 and stores the integrated analysis target information 333 in the storage device 313.

P49. The analyst analyzes the integrated analysis target information 333 using the analyst terminal 311 and transmits an analysis result to the server 312. The server 312 stores the received analysis result in the analysis result DB 332.

P50. The server 312 provides the user terminal 306 to the user terminal 308 with the analysis result, and a university, a research institution, and a pharmaceutical company obtains the analysis result using the user terminal 306 to the user terminal 308.

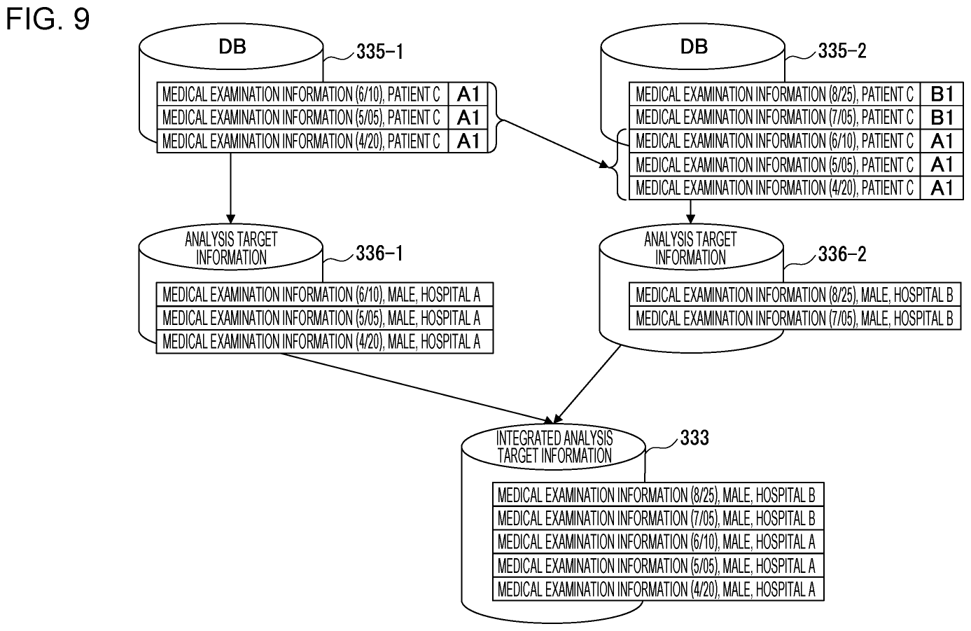

FIG. 9 illustrates an example of the processing that generates integrated analysis target information. Before the patient C of the hospital A moves to the hospital B, the VM 334-1 adds the medical institution ID “A1” of the hospital A to the medical examination information (4/20) of the patient C, the medical examination information (5/05), and the medical examination information (6/10) and stores the information in the DB 335-1. When the patient C moves from the hospital A to the hospital B, the medical examination information of the patient C in the DB 335-1 is copied to the DB 335-2.

After the hospital B received the patient C, the VM 334-2 adds the medical institution ID “B1” of the hospital B to the medical examination information (7/05) of the patient C and medical examination information (8/25), and stores the information in the DB 335-2.

Next, the VM 334-1 extracts only the medical examination information to which the medical institution ID “A1” is added from the medical examination information in the DB 335-1 and conceals the extracted medical examination information so as to generate analysis target information 336-1. The VM 334-2 extracts only the medical examination information to which the medical institution ID “B1” is added from the medical examination information in DB 335-2 and conceals the extracted medical examination information so as to generate analysis target information 336-2. The aggregation device 314 merges the analysis target information 336-1 and the analysis target information 336-2 to generate integrated analysis target information 333.

In this manner, the medical examination information in each hospital is added with its medical institution ID, and when medical examination information is copied between hospitals, the medical examination information is copied with the medical institution ID of the original hospital added. Thereby, it is possible for the transfer destination hospital to distinguish the medical examination information of a patient in the original hospital and the medical examination information of the same patient in the transfer destination hospital.

In the transfer destination hospital, by extracting only the medical examination information to which its medical institution ID is added, the copied medical examination information is excluded, and thus the medical examination information of the same patient is no longer redundantly collected from the two places, the original hospital and the transfer destination hospital. Accordingly, it becomes possible to provide an accurate analysis result for the collected medical examination information to a university, a research institution, and a pharmaceutical company.

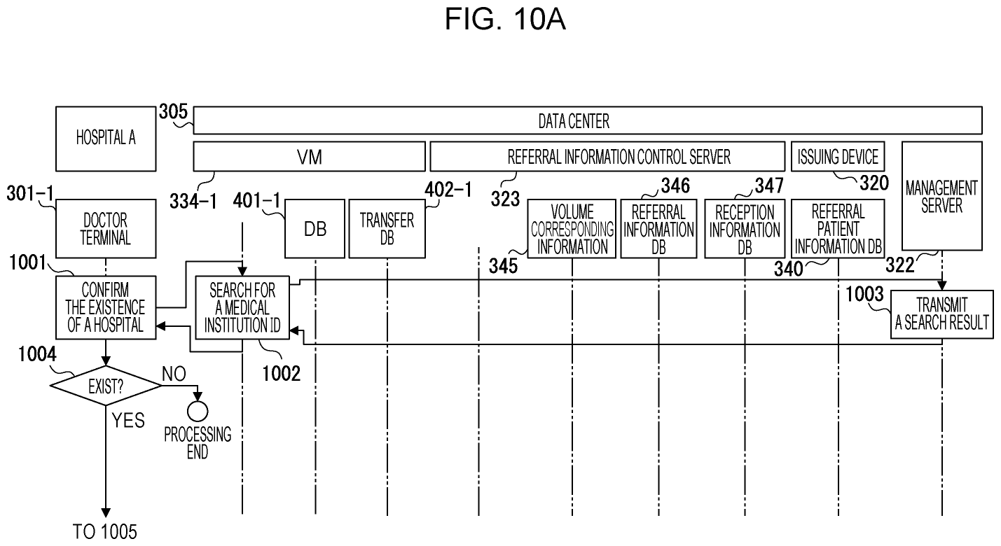

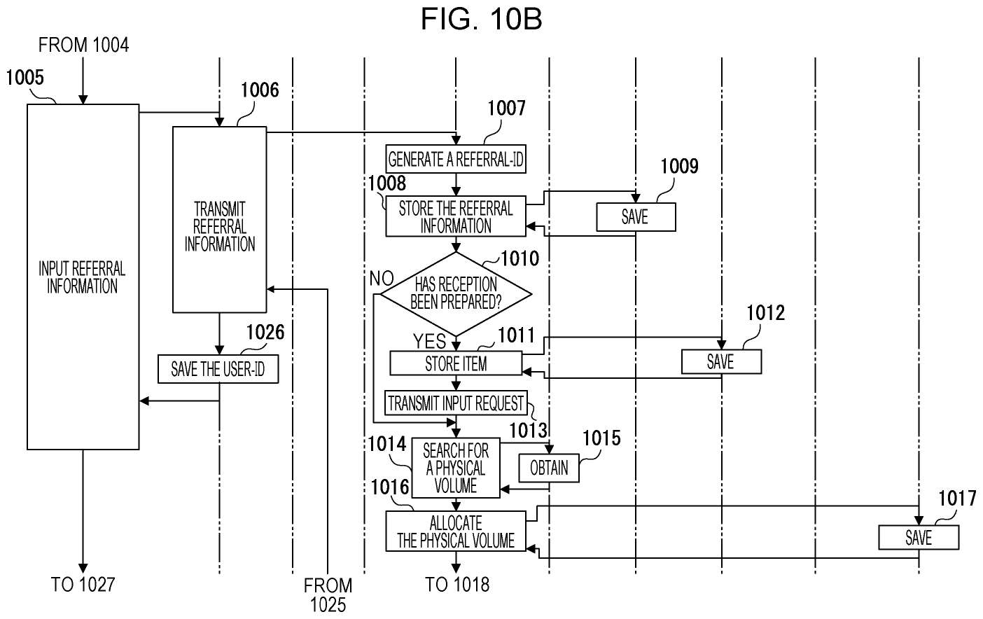

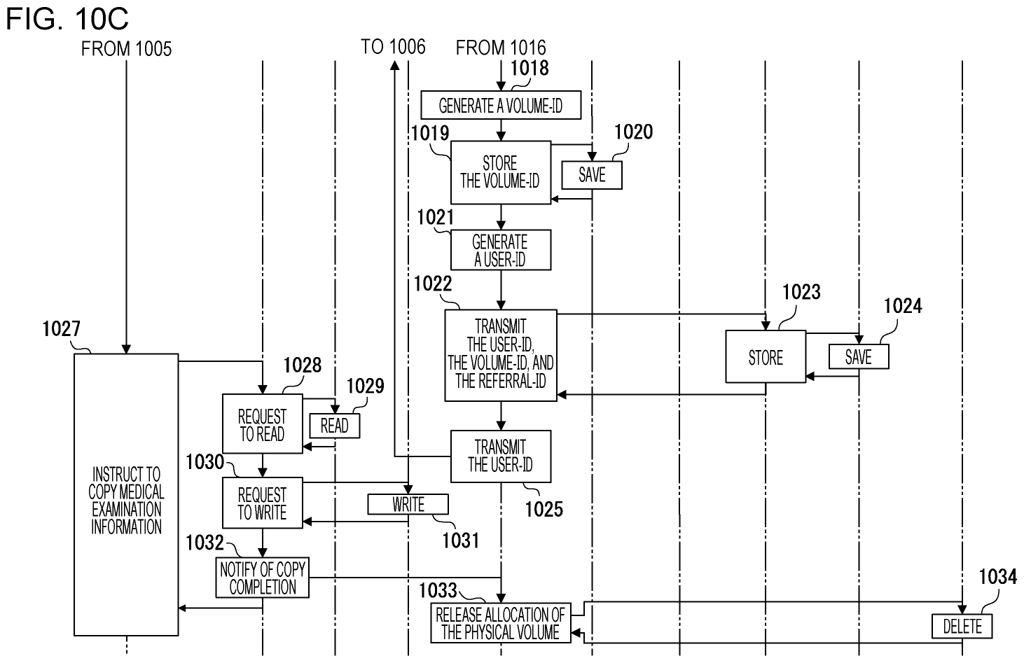

Next, a more detailed description will be given of the operation of the information processing system in FIGS. 3A and 3B with reference to FIG. 10A to FIG. 18B.

FIGS. 10A, 10B, and 10C illustrate an example of a referral sequence in which a doctor in the hospital A gives the patient a referral to the hospital B. First, the doctor in the hospital A inputs the hospital name of the hospital B in the VM 334-1 in order to confirm the existence of a referral destination hospital B using the doctor terminal 301-1 (step 1001). The VM 334-1 transmits a medical institution ID corresponding to the input hospital name to the management server 322 to search for its medical institution ID (step 1002).

The management server 322 checks whether or not the received medical institution ID is the medical institution ID of an existing VM 334-i and transmits a search result to the VM 334-1 (step 1003). The VM 334-1 transmits the received search result to the doctor terminal 301-1, and the doctor terminal 301-1 determines whether or not the hospital B exists based on the received search result (step 1004). If the hospital B does not exist (step 1004, NO), the doctor terminal 301-1 terminates the processing.

If the hospital B exists (step 1004, YES), the doctor of the hospital A inputs the referral information of the patient to the VM 334-1 using the doctor terminal 301-1 (step 1005). The VM 334-1 transmits the input referral information to the referral information control server 323 (step 1006).

The allocation unit 342 of the referral information control server 323 receives referral information, generates a referral-ID for the patient (step 1007), adds the referral-ID to the received referral information, and records the referral information in the referral information DB 346 (step 1008). The referral information DB 346 saves the referral information including the referral-ID (step 1009).

Next, the allocation unit 342 checks whether or not the referral destination hospital B makes reception preparations for the patient (step 1010). If the hospital B does not make reception preparations (step 1010, NO), the referral information control server 323 performs the processing of step 1014 and after that.

If the hospital B makes reception preparations (step 1010, YES), the allocation unit 342 records the items of the reception information with the referral-ID in the reception information DB 347 (step 1011). The reception information DB 347 saves the items of the reception information in association with the referral-ID (step 1012). The allocation unit 342 transmits an input request of the reception information with the referral-ID to the medical office terminal 302-2 of the hospital B (step 1013).

Next, the allocation unit 342 searches the volume corresponding information 345 for an unused physical volume 338-j (step 1014) and obtains the location information of an unused physical volume 338-1 (step 1015). The allocation unit 342 allocates a physical volume 338-1 to the transfer DB 402-1 of the VM 334-1 and transmits an allocation result to the management server 322 (step 1016). The management server 322 saves the association relationship between the transfer DB 402-1 of the VM 334-1 and the physical volume 338-1 (step 1017).

Next, the allocation unit 342 generates a volume-ID for the physical volume 338-1 (step 1018) and records the generated volume-ID in association with the location information of the physical volume 338-1 in the volume corresponding information 345 (step 1019). The allocation unit 342 saves the volume corresponding information 345 (step 1020).

Next, the allocation unit 342 generates a user-ID of the patient (step 1021) and transmits the user-ID, the volume-ID, and the referral-ID to the issuing device 320 (step 1022). The issuing device 320 generates referral patient information including the received user-ID, the volume-ID, and the referral-ID and records the referral patient information in the referral patient information DB 340 (step 1023). The referral patient information DB 340 stores the referral patient information (step 1024).

Next, the control unit 343 of the referral information control server 323 transmits the user-ID to the VM 334-1 and notifies of the allocation completion of the physical volume 338-1 (step 1025). The VM 334-1 saves the received user-ID and notifies the doctor terminal 301-1 of the allocation completion of the physical volume 338-1 (step 1026).

Next, the doctor of the hospital A instructs the VM 334-1 to copy the medical examination information using the doctor terminal 301-1 (step 1027). The VM 334-1 transmits a read request for reading the medical examination information of the patient, which is the copy target, to the DB 401-1 (step 1028) and reads the medical examination information of the patient from the DB 335-1 which is allocated to the DB 401-1 (step 1029).

Next, the VM 334-1 transmits a write request for writing the medical examination information of the patient to the transfer DB 402-1 (step 1030) and writes the medical examination information of the patient in the physical volume 338-1, which is allocated to the transfer DB 402-1 (step 1031). Thereby, the medical examination information of the patient is copied from the DB 335-1 to the physical volume 338-1. The VM 334-1 notifies the referral information control server 323 of the copy completion (step 1032).

The allocation unit 342 of the referral information control server 323 releases the allocation of the physical volume 338-1 to the transfer DB 402-1 and notifies the management server 322 of the allocation release (step 1033). The management server 322 deletes the association relationship between the transfer DB 402-1 of the VM 334-1 and the physical volume 338-1 (step 1034).

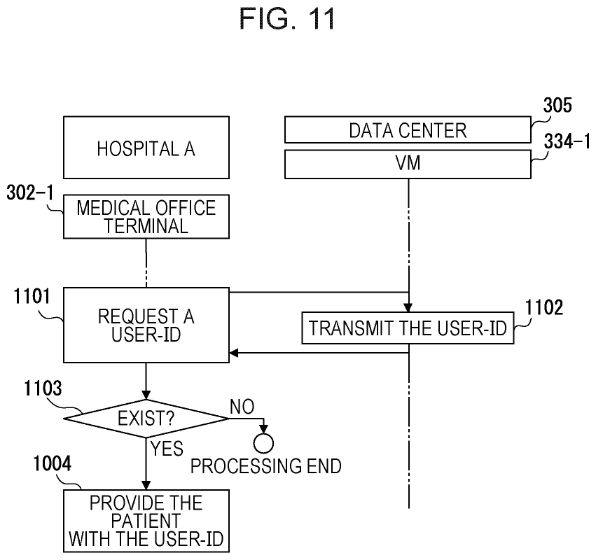

FIG. 11 illustrates an example of the user-ID providing sequence to provide a user-ID to a patient. First, a clerk of the hospital A requests a user-ID of the patient from the VM 334-1 using the medical office terminal 302-1 (step 1101). The VM 334-1 transmits the user-ID received from the referral information control server 323 to the medical office terminal 302-1 (step 1102). If the VM 334-1 does not hold the requested patient user-ID, the VM 334-1 notifies the medical office terminal 302-1 of nonexistence of the user-ID.

The medical office terminal 302-1 determines whether or not the user-ID of the patient exists based on the information received from the VM 334-1 (step 1103). If the user-ID does not exist (step 1103, NO), the processing is terminated. If the user-ID exists (step 1103, YES), the clerk of the hospital A provides the patient of the user-ID received by the medical office terminal 302-1 (step 1104).

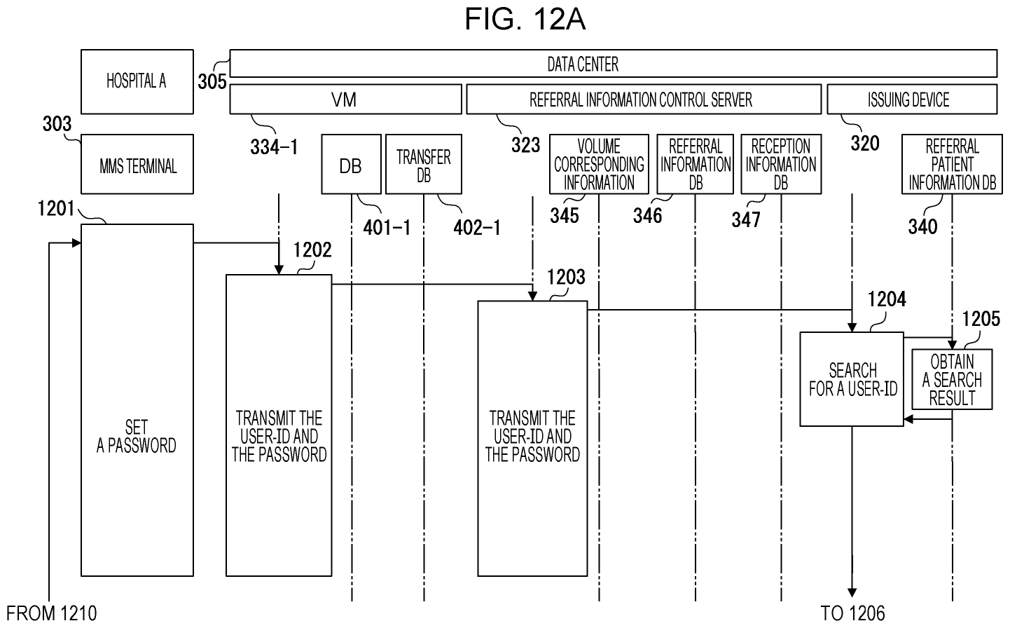

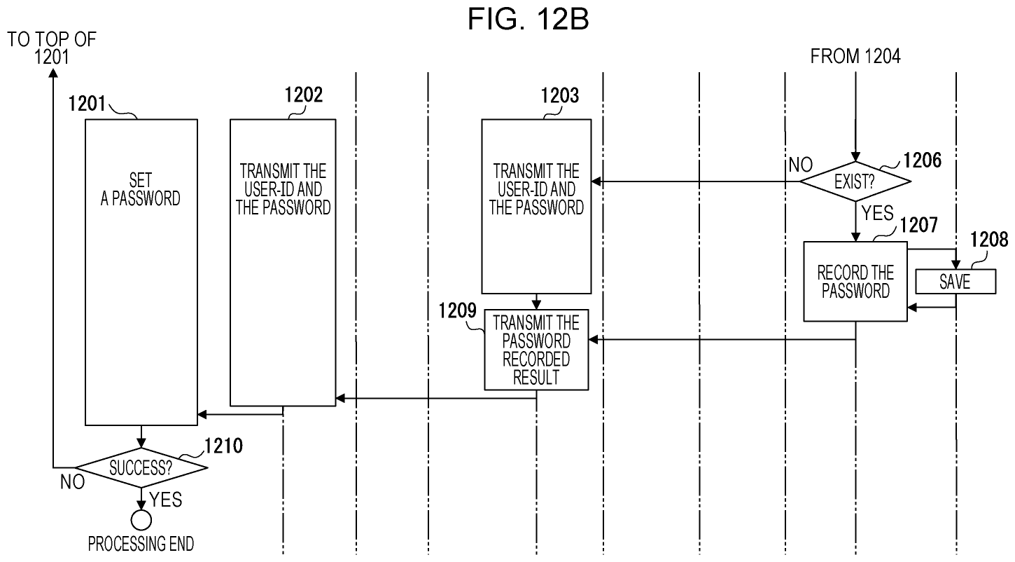

FIGS. 12A and 12B illustrate an example of the password setting sequence for a patient to set a password for his or her user-ID. First, the patient sets a password for the user-ID using the MMS terminal 303 and the MMS terminal 303 transmits the set password to the VM 334-1 (step 1201).

The VM 334-1 transmits the user-ID and the password to the referral information control server 323 (step 1202), and the referral information control server 323 transmits the received user-ID and password to the issuing device 320 (step 1203).

The issuing device 320 searches the referral patient information DB 340 for the received user-ID (step 1204) and obtains a search result (step 1205). The issuing device 320 determines whether or not the received user-ID exists in the referral patient information DB 340 (step 1206). If the user-ID does not exist (step 1206, NO), the issuing device 320 notifies the referral information control server 323 of nonexistence of the user-ID.

If the user-ID exists (step 1206, YES), the issuing device 320 records the hash value of the received password in the referral patient information corresponding to the user-ID in the referral patient information DB 340 (step 1207). The referral patient information DB 340 saves the referral patient information (step 1208). The issuing device 320 notifies the referral information control server 323 that the password has been recorded.

The referral information control server 323 transmits the recorded result of the password to the VM 334-1, and the VM 334-1 transmits the received recorded result to the MMS terminal 303. The MMS terminal 303 determines whether or not the setting a password has been successful based on the received recorded result (step 1210). If the setting a password was unsuccessful (step 1210, NO), the processing of step 1201 and after that is repeated. If the setting a password was successful (step 1210, YES), the MMS terminal 303 terminates the processing.

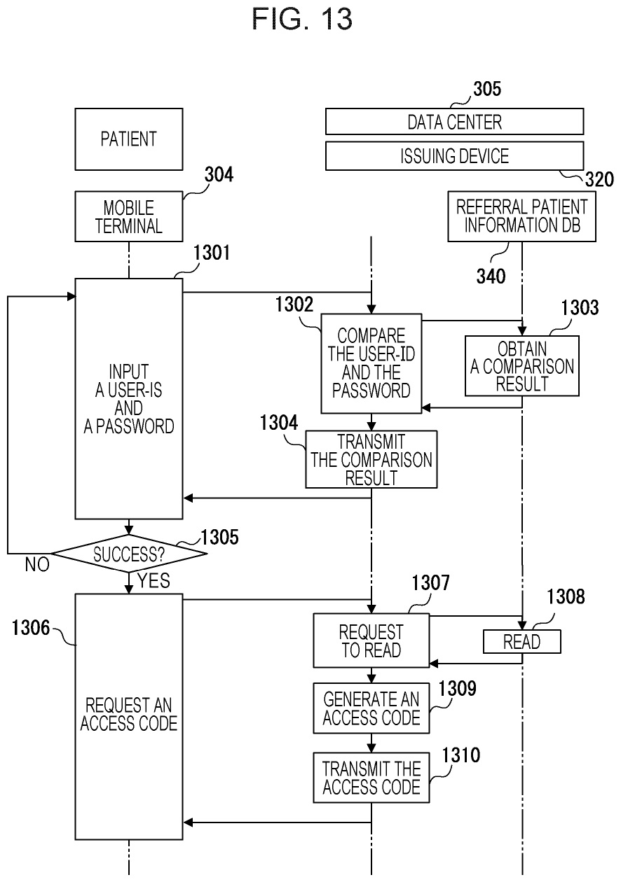

FIG. 13 illustrates an example of access code acquisition sequence for the patient to obtain an access code. First, the patient inputs a user-ID and a password using the mobile terminal 304, and the mobile terminal 304 transmits the input user-ID and password to the issuing device 320 (step 1301).

The issuing device 320 compares the received user-ID and password with the referral patient information in the referral patient information DB 340 (step 1302) and obtains a comparison result (step 1303). The issuing device 320 transmits the comparison result to the mobile terminal 304 (step 1304).

The mobile terminal 304 determines whether or not the login to the referral service has been successful based on the received comparison result (step 1305). If the login has been unsuccessful (step 1305, NO), the processing of step 1301 and after that is repeated. If the login has been successful (step 1305, YES), the mobile terminal 304 requests an access code from the issuing device 320 (step 1306).

The issuing device 320 transmits a read request for reading a referral-ID and a volume-ID to the referral patient information DB 340 (step 1307), and reads a referral-ID and a volume-ID from the referral patient information corresponding to the user-ID (step 1308). The issuing device 320 generates an access code from the read referral-ID and volume-ID (step 1309) and transmits the generated access code to the mobile terminal 304 (step 1310). Thereby, an access code is given to the patient.

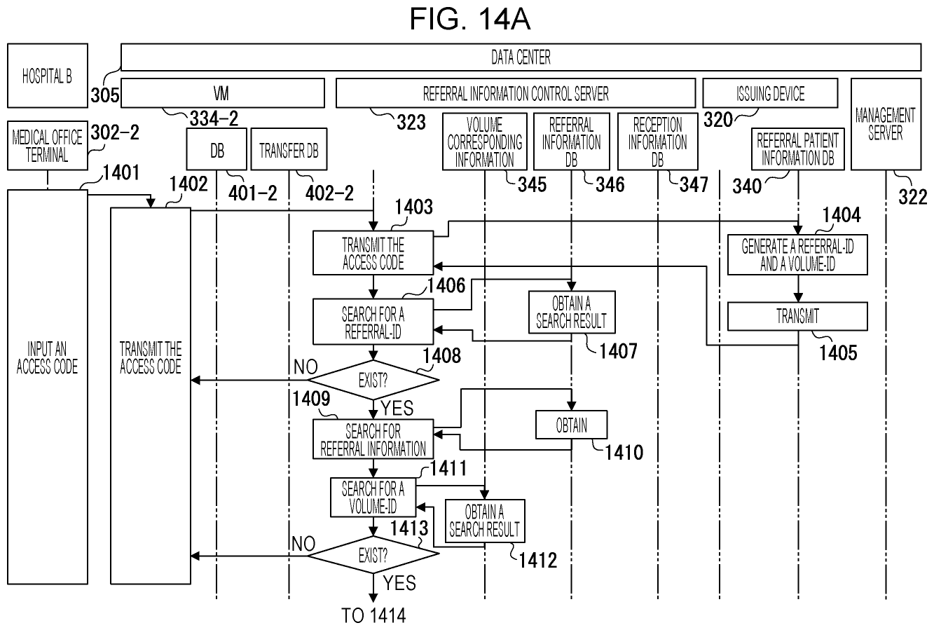

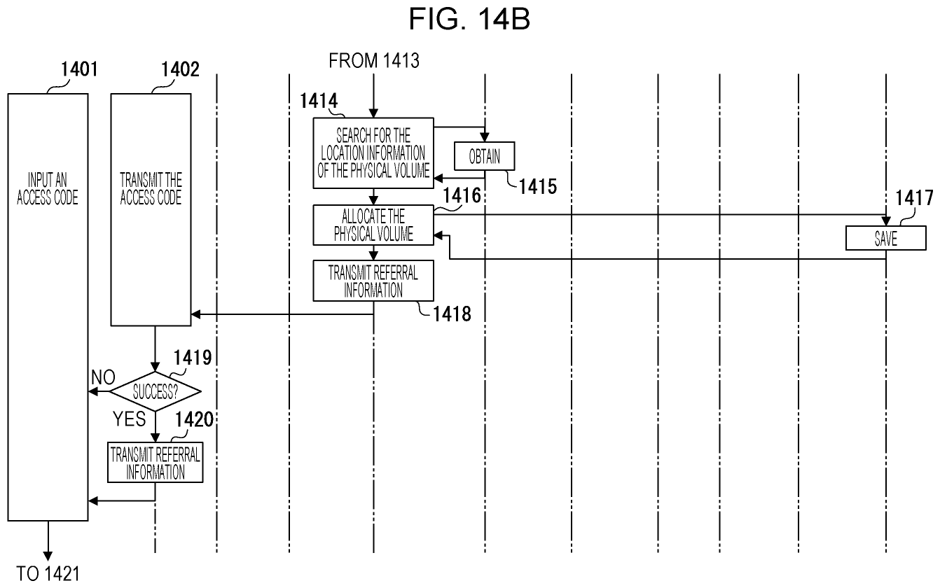

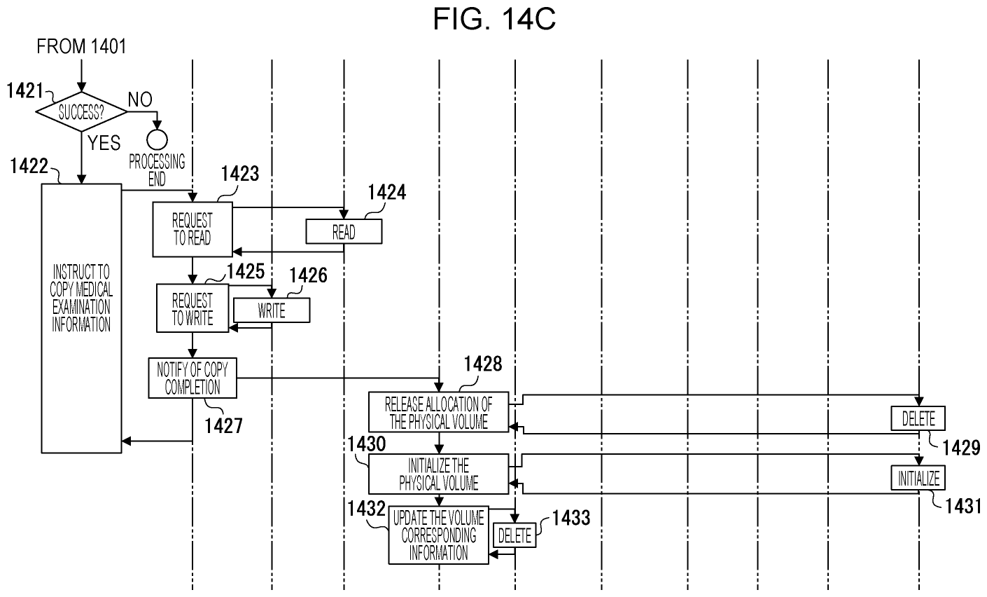

FIGS. 14A, 14B, and 14C illustrate an example of the reception sequence for the hospital B to receive a patient. First, a clerk of the hospital B inputs the access code presented by the patient to the VM 334-2 using the medical office terminal 302-2 (step 1401). The VM 334-2 transmits the input access code to the referral information control server 323 (step 1402). The allocation unit 342 of the referral information control server 323 receives the access code and transmits the received access code to the issuing device 320 (step 1403).

The issuing device 320 generates a referral-ID and a volume-ID from the received access code (step 1404) and transmits the generated referral-ID and volume-ID to the referral information control server 323 (step 1405).

The allocation unit 342 searches the referral information DB 346 for the received referral-ID (step 1406) and obtains a search result (step 1407). The allocation unit 342 determines whether or not the received referral-ID exists in the referral information DB 346 (step 1408). If the referral-ID does not exist (step 1408, NO), the allocation unit 342 notifies the VM 334-2 of nonexistence of the referral-ID.

If the referral-ID exists (step 1408, YES), the allocation unit 342 searches the referral information DB 346 for the referral information corresponding to the referral-ID (step 1409) and obtains the referral information (step 1410).

Next, the allocation unit 342 searches the volume corresponding information 345 for the volume-ID included in the obtained referral information (step 1411) and obtain a search result (step 1412). The allocation unit 342 determines whether or not the volume-ID exists in the volume corresponding information 345 (step 1413). If the volume-ID does not exist (step 1413, NO), the allocation unit 342 notifies the VM 334-2 of nonexistence of the volume-ID.

If the referral-ID exists (step 1413, YES), the allocation unit 342 searches the volume corresponding information 345 for the location information of the physical volume 338-j corresponding to the volume-ID (step 1414). The allocation unit 342 obtains the location information of the physical volume 338-1 (step 1415).

Next, the allocation unit 342 allocates the physical volume 338-1 to the transfer DB 402-2 of the VM 334-2 and transmits an allocation result to the management server 322 (step 1416). The management server 322 saves the association relationship between the transfer DB 402-2 of the VM 334-2 and the physical volume 338-1 (step 1417).

Next, the control unit 343 of the referral information control server 323 transmits the referral information to the VM 334-2 and notifies of the allocation completion of the physical volume 338-1 (step 1418).

The VM 334-2 determines whether or not the allocation of the physical volume 338-1 has been successful based on the notification received from the referral information control server 323 (step 1419). If the allocation has not been successful (step 1419, NO), the VM 334-2 notifies the medical office terminal 302-2 that the access code is incorrect.

If the allocation has been successful (step 1419, YES), the VM 334-2 transmits the received referral information to the medical office terminal 302-2, and the medical office terminal 302-2 displays the received referral information on its screen (step 1420). Thereby, it is possible for the clerk of the hospital B to confirm the information, such as the name of the doctor of the hospital A, who is a referrer, a medical department suitable for treatment of the patient, the disease name of the patient, and the like.

Next, the medical office terminal 302-2 determines whether or not an access to the referral information has been successful based on the information received from the VM 334-2 (step 1421). If the access has not been successful (step 1421, NO), the medical office terminal 302-2 terminates the processing.

If the access has been successful (step 1421, YES), the clerk of the hospital B instructs to copy the medical examination information to the VM 334-2 using the medical office terminal 302-2 (step 1422). The VM334-2 transmits a read request for reading the medical examination information of the patient to the transfer DB 402-2 (step 1423) to read the medical examination information of the patient from the physical volume 338-1 allocated to the transfer DB 402-2 (step 1424).

Next, the VM 334-2 transmits a write request for writing the medical examination information of the patient to the DB 401-2 (step 1425) to write the medical examination information of the patient in the DB 335-2 allocated to the DB 401-2 (step 1426). Thereby, the medical examination information of the patient is copied from the physical volume 338-1 to the DB 335-2. The VM 334-2 notifies the referral information control server 323 of the copy completion (step 1427).

The allocation unit 342 of the referral information control server 323 releases the allocation of the physical volume 338-1 to the transfer DB 402-2 and notifies the management server 322 of the allocation release (step 1428). The management server 322 deletes the association relationship between the transfer DB 402-2 of the VM 334-2 and the physical volume 338-1 (step 1429).

Next, the allocation unit 342 transmits an instruction to initialize the physical volume 338-1 to the management server 322 (step 1430), and the management server 322 initializes the management information of the physical volume 338-1 (step 1431).

Next, the allocation unit 342 performs update processing of the volume corresponding information 345 (step 1432) and deletes the volume-ID received from the issuing device 320 from the volume corresponding information 345 (step 1433). Thereby, the physical volume 338-1 returns to an unused state in the volume corresponding information 345, and thus the medical examination information in the physical volume 338-1 is discarded.

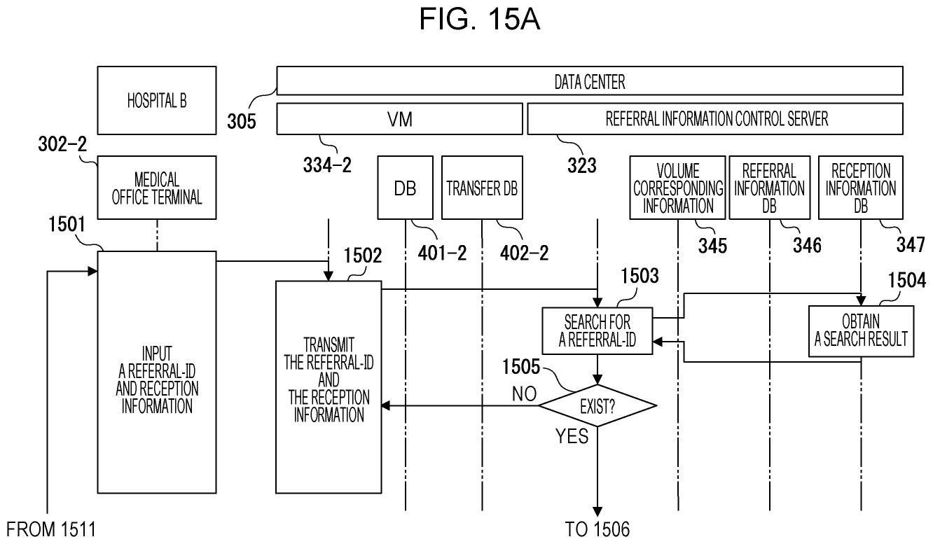

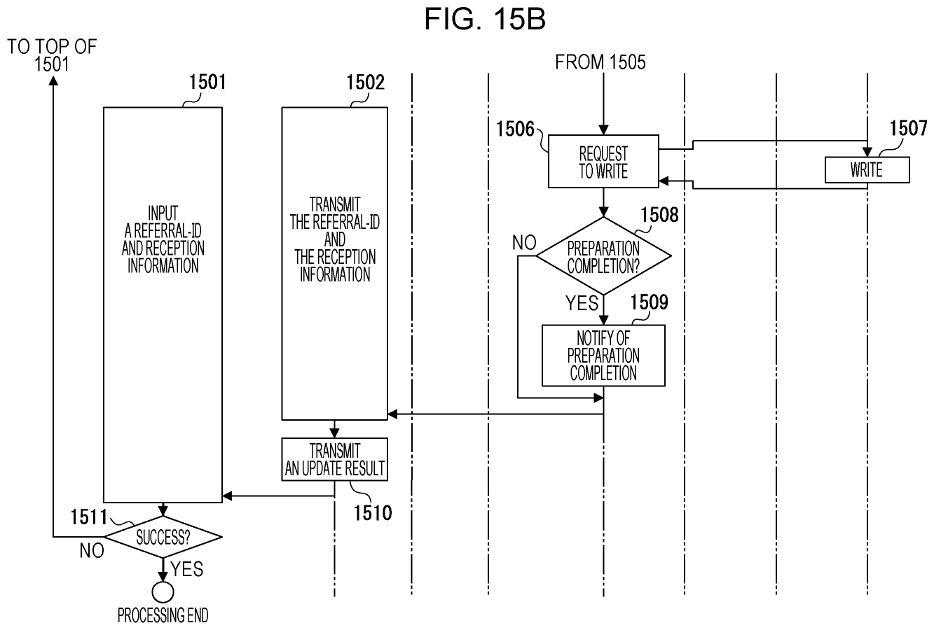

FIGS. 15A and 15B illustrate an example of the reception information generation sequence for the hospital B to input the reception information for receiving a patient. The reception information generation sequence in FIGS. 15A and 15B is executed when the hospital B receives an input request of reception information from the referral information control server 323 or when the hospital B updates the reception information.

First, the clerk of the hospital B inputs the referral-ID and the reception information of the patient in the VM 334-2 using the medical office terminal 302-2 (step 1501). The VM 334-2 transmits the input referral-ID and reception information to the referral information control server 323 (step 1502).

The allocation unit 342 of the referral information control server 323 searches the reception information DB 347 for the received referral-ID (step 1503) and obtains a search result (step 1504). The allocation unit 342 determines whether or not the received referral-ID exists in the reception information DB 347 (step 1505). If the referral-ID does not exist (step 1505, NO), the allocation unit 342 notifies the VM 334-2 of an update result indicating the update failure of the reception information.

If the referral-ID exists (step 1505, YES), the allocation unit 342 transmits a write request to write the reception information to the reception information DB 347 (step 1506) to write the reception information in the reception information DB 347 (step 1507). When the hospital B receives an input request of reception information, the contents of new reception information is written in the reception information DB 347, and when the hospital B updates the reception information, the contents of the update-target reception information is updated.

Next, the allocation unit 342 refers to the item of the state included in the written reception information and determines whether or not the reception preparations for the patient have been completed (step 1508). If the state indicates preparation completion, a determination is made that the reception preparations have been completed, whereas if the state indicates waiting, a determination is made that the reception preparations have not been completed.

If the reception preparations have not been completed (step 1508, NO), the allocation unit 342 notifies the VM 334-2 of the update result indicating the update completion of the reception information. If the reception preparations have been completed (step 1508, YES), the allocation unit 342 notifies the VM 334-2 of the update result indicating the preparation completion (step 1509). The VM 334-2 transmits the notified update result to the medical office terminal 302-2 (step 1510).

The medical office terminal 302-2 determines whether or not update of the reception information has been successful based on the received update result (step 1511). If the update of the reception information has not been successful (step 1511, NO), the medical office terminal 302-2 repeats the processing of step 1501 and after that. If the update of the reception information has been successful (step 1511, YES), the medical office terminal 302-2 terminates the processing.

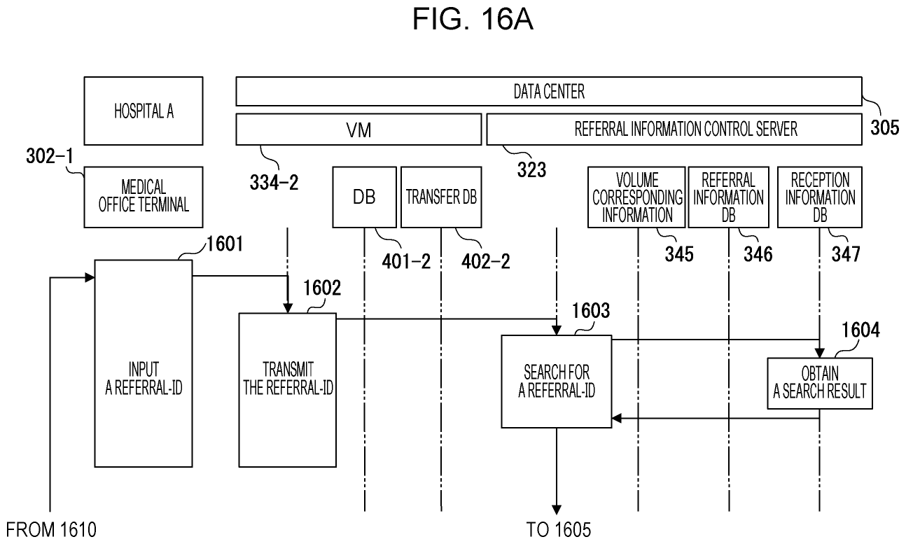

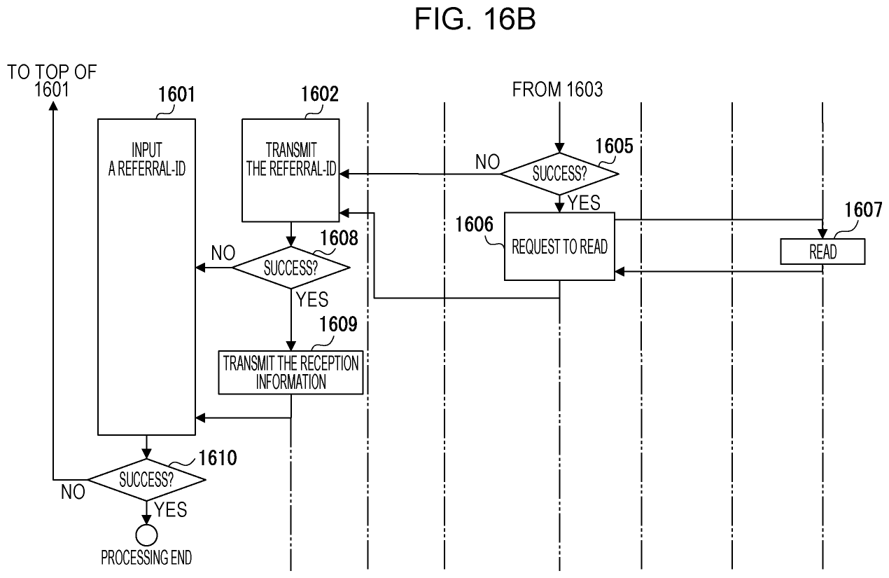

FIGS. 16A and 16B illustrate an example of the reception information confirmation sequence for the hospital A to confirm the state of the reception preparations for the patient. First, a clerk of the hospital A inputs a referral-ID in the VM 334-2 using the medical office terminal 302-1 (step 1601). The VM 334-2 transmits the input referral-ID to the referral information control server 323 (step 1602).

The allocation unit 342 of the referral information control server 323 searches the reception information DB 347 for the received referral-ID (step 1603) to obtain a search result (step 1604). The allocation unit 342 determines whether or not the received referral-ID exists in the reception information DB 347 (step 1605). If the referral-ID does not exist (step 1605, NO), the allocation unit 342 transmits the processing result indicating the failure of obtaining the reception information to the VM 334-2.

If the referral-ID exists (step 1605, YES), the allocation unit 342 transmits a read request to read the reception information to the reception information DB 347 (step 1606) to read the reception information from the reception information DB 347 (step 1607). The allocation unit 342 transmits the read reception information to the VM 334-2 as a processing result.

The VM 334-2 determines whether or not obtaining the reception information has been successful based on the received processing result (step 1608). If obtaining the reception information has not been successful (step 1608, NO), the VM 334-2 notifies the medical office terminal 302-1 of a failure of the confirmation of the reception information. If obtaining the reception information has been successful (step 1608, YES), the VM 334-2 transmits the reception information to the medical office terminal 302-1, and the medical office terminal 302-1 displays the received reception information on the screen (step 1609). Thereby, it is possible for the clerk of the hospital A to confirm whether or not the reception preparations for the patient have been completed.

Next, the medical office terminal 302-1 determines whether or not the confirmation of the reception information has been successful based on the information received from the VM 334-2 (step 1610). If the confirmation of the reception information has not been successful (step 1610, NO), the medical office terminal 302-1 repeats the processing of step 1601 and after that. If the confirmation of the reception information has been successful (step 1610, YES), the medical office terminal 302-1 terminates the processing.

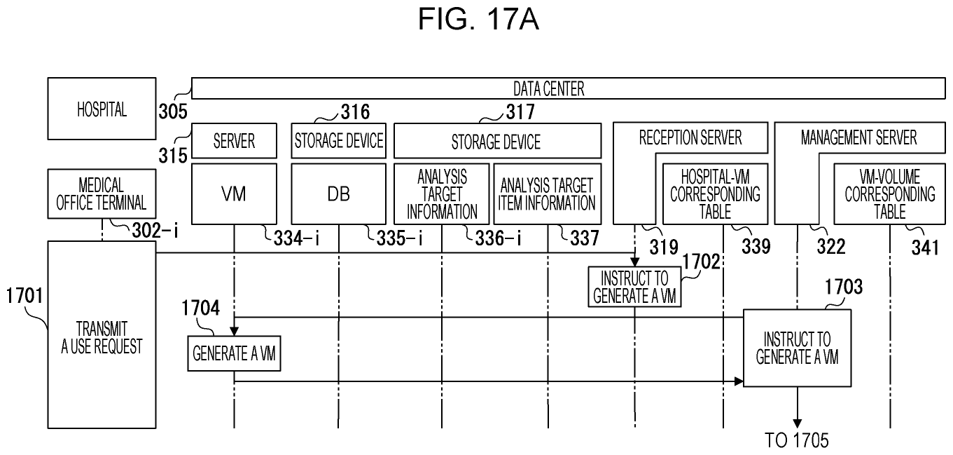

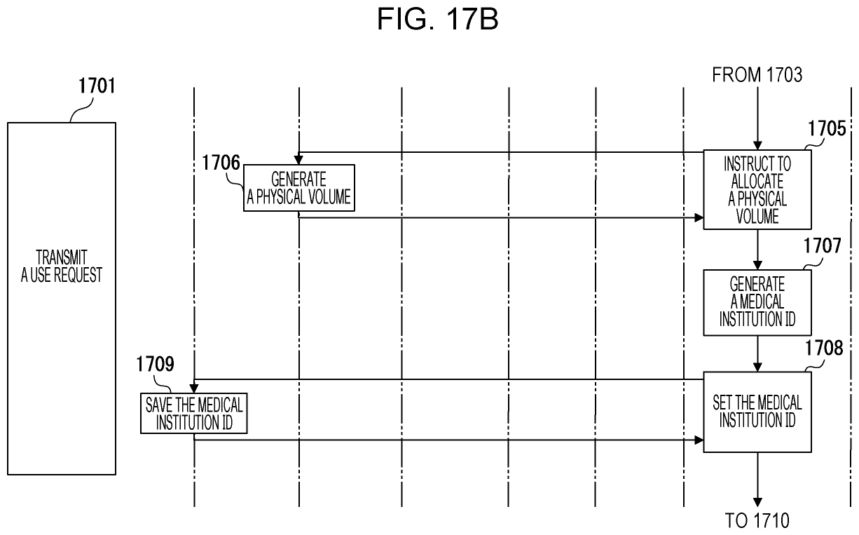

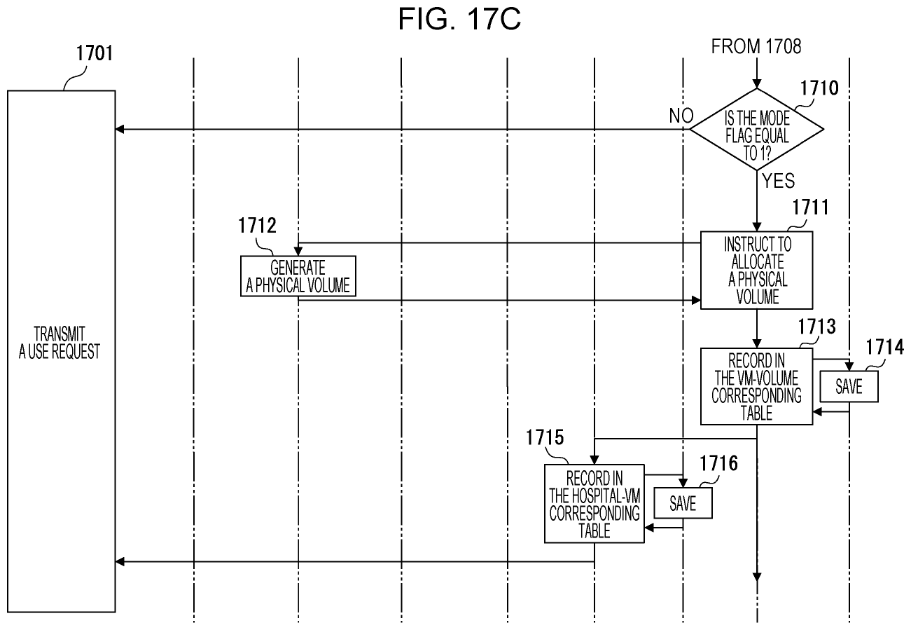

FIGS. 17A, 17B, and 17C illustrate an example of the volume allocation sequence to allocate a physical volume to the VM 334-i when each hospital applies for use of the information processing system. First, a clerk of the i-th hospital transmits a use application to the reception server 319 using the medical office terminal 302-i (step 1701). The use application includes information of whether or not the medical examination information is allowed to be provided to an analyst.

The reception server 319 instructs the management server 322 to generate a VM (step 1702). The management server 322 instructs the server 315 to generate a VM (step 1703), and the server 315 generates a VM 334-i (step 1704). When a use application allows the medical examination information to be provided, the server 315 sets the mode flag of the VM 334-i to a logic “1”,whereas when a use application does not allow the medical examination information to be provided, the server 315 sets the mode flag to a logic “0”.

Next, the management server 322 instructs the storage device 316 to allocate a physical volume (step 1705), and the storage device 316 generates a physical volume of the DB 335-i (step 1706).

Next, the management server 322 generates a medical institution ID that indicates the i-th hospital (step 1707) and transmits the generated medical institution ID to the VM 334-i (step 1708). The VM 334-i saves the received medical institution ID (step 1709).

Next, the management server 322 checks the mode flag of the VM 334-i (step 1710). If the mode flag is the logic “0” (step 1710, NO), the management server 322 notifies the medical office terminal 302-i of the completion of generating a VM.

If the mode flag is the logic “1” (step 1710, YES), the management server 322 instructs the storage device 317 to allocate a physical volume (step 1711). The storage device 317 generates a physical volume for the analysis target information 336-i (step 1712).

Next, the management server 322 records the association relationship between the VM 334-i and the physical volume of the analysis target information 336-i in the VM-volume corresponding table 341 (step 1713). The association relationship is saved in the VM-volume corresponding table 341 (step 1714).

Next, the management server 322 transmits the generated medical institution ID to the reception server 319, and the reception server 319 records the association relationship between the medical institution ID and the VM 334-i in the hospital-VM corresponding table 339 (step 1715). The association relationship is saved in the hospital-VM corresponding table 339 (step 1716). The reception server 319 notifies the medical office terminal 302-i of the completion of the VM generation.

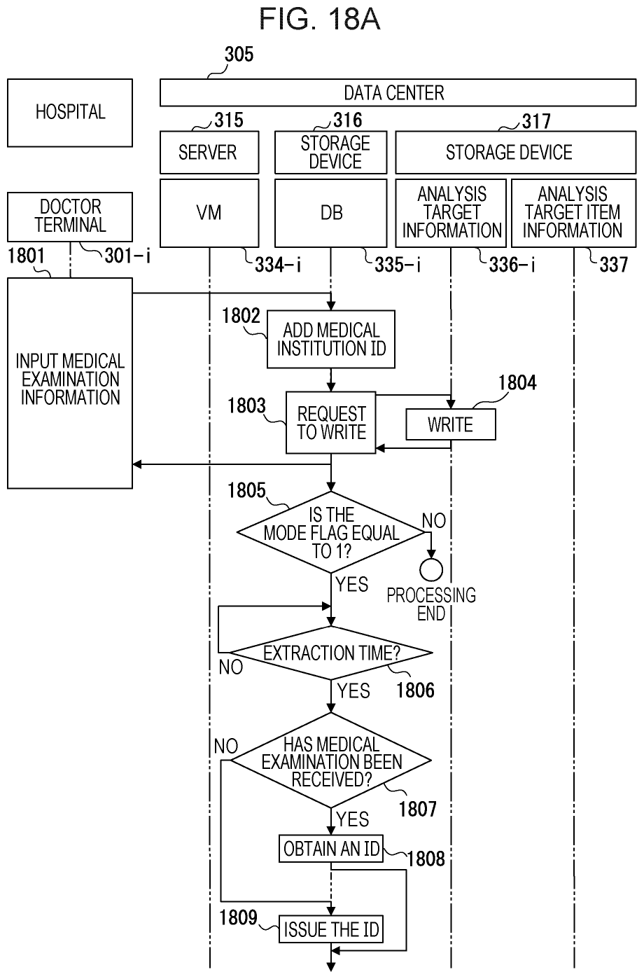

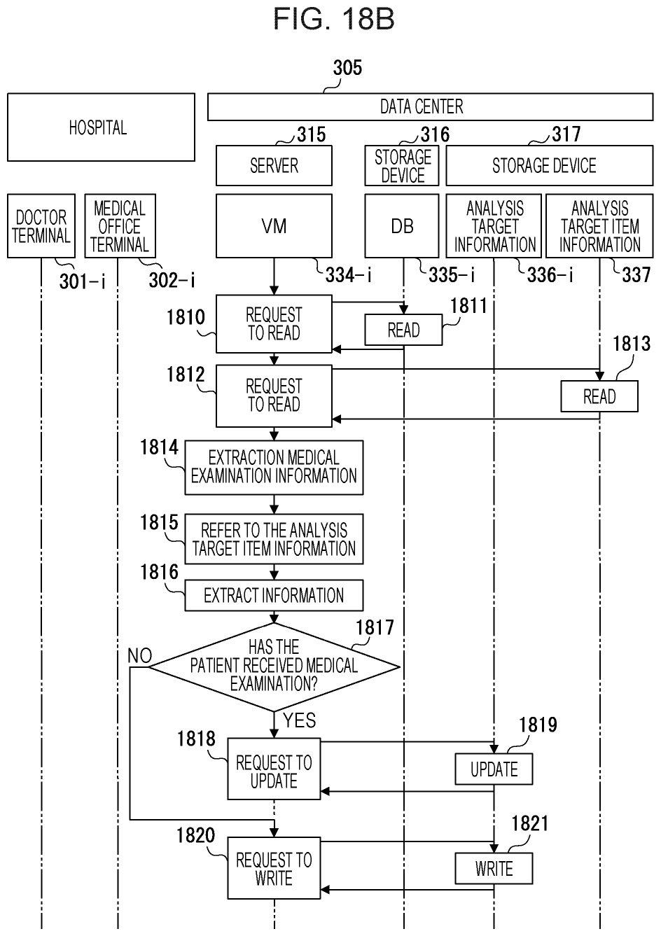

FIGS. 18A and 18B illustrate an example of the information extraction sequence to extract analysis target information from the medical examination information of a patient at each hospital. First, a doctor of the i-th hospital inputs the medical examination information of a patient in the VM 334-i using the terminal 301-i (step 1801).

Next, the VM 334-i adds a medical institution ID to the input medical examination information (step 1802). The VM 334-i transmits a write request for writing the medical examination information to the DB 335-i (step 1803) to write the medical examination information with a medical institution ID in the DB 335-i (step 1804).

Next, the VM 334-i checks the mode flag (step 1805). If the mode flag is the logic “0” (step 1805, NO), the processing is terminated. If the mode flag is the logic “1” (step 1805, YES), the VM 334-i checks whether or not the extraction time scheduled in advance has come (step 1806).

If the extraction time has not come yet (step 1806, NO), the VM 334-i repeats the processing of step 1806. If the extraction time has come (step 1806, YES), the VM 334-i refers to a holding patient list and checks whether or not the patient of the medical examination information written in the DB 335-i has received a medical examination in the past (step 1807). If the name of the medical examination information is included in the patient list, the VM 334-i determines that the patient already has received a medical examination. If the name of the medical examination information is not included in the patient list, the VM 334-i determines that the patient has not received a medical examination.

If the patient has not received a medical examination (step 1807, NO), the VM 334-i issues a new ID and records the patient name and the issued ID in the patient list in association with each other (step 1809). The VM 334-i gives the issued ID to the medical examination information written in the DB 335-i.

If the patient has received a medical examination (step 1807, YES), the VM 334-i obtains the ID corresponding to the patient name from the patient list (step 1808). The VM 334-i gives the obtained ID to the medical examination information written in the DB 335-i.

Next, the VM 334-i transmits a read request for reading the medical examination information to the DB 335-i (step 1810) to read the medical examination information that has been written after the previous extraction time from the DB 335-i as the difference of the medical examination information (step 1811).

Next, the VM 334-i transmits a read request for reading analysis target item information 337 to the storage device 317 (step 1812) to read the analysis target item information 337 from the storage device 317 (step 1813). The VM 334-i deletes the medical examination information to which a medical institution ID different from the medical institution ID of the i-th hospital is added out of the medical examination information read from the DB 335-i so as to extract only the medical examination information to which the medical institution ID of the i-th hospital is added (step 1814).

Next, the VM 334-i refers to the analysis target item information 337 (step 1815) and extract information of the analysis target item from the extracted medical examination information (step 1816). At this time, it is possible for the VM 334-i to produce information from which an individual is not identified by extracting the information of the non-analysis target item once and performing concealing processing.

Next, the VM 334-i checks the determination result of step 1807 once again (step 1817). If the patient has not received a medical examination (step 1817, NO), the VM 334-i transmits a write request for writing extracted information to the storage device 317 (step 1820). The storage device 317 writes the received information as the analysis target information 336-i of a new patient (step 1821).

If the patient has received a medical examination (step 1817, YES), the VM 334-i transmits an update request for updating the analysis target information 336-i including the same ID as the ID obtained from the patient list to the storage device 317 (step 1818). The storage device 317 updates the analysis target information 336-i with the received information (step 1819). For example, if the information extracted by the VM 334-i corresponds to a prescription, an examination result, or a disease name, the extracted information is added to the information of the item included in the existing analysis target information 336-i. Alternatively, the existing analysis target information 336-i is rewritten by the extracted information.

The configurations of the information processing systems in FIG. 1 and FIGS. 3A and 3B are only examples, and some of the components may be omitted or changed in accordance with the use or the conditions of the information processing system. For example, the doctor terminal 301-i, the medical office terminal 302-i, and the MMS terminal 303 may be installed in the other medical institutions, such as a clinic, a geriatric health services facility, or the like. When the referral information control server 323 issues an access code instead of the issuing device 320, it is possible to omit the issuing device 320. The VM 334-2 may be operated in a server different from the server 315.

The logical volumes illustrated in FIG. 4 are only examples, the other logical volumes may be used depending on the use or the conditions of the information processing system.

The flowchart in FIG. 2 and the operation sequence in FIG. 10A to FIG. 18B are only examples, and a part of the processing may be omitted or changed in accordance with the configuration of the information processing system or the conditions. For example, an access code including only a volume-ID may be used instead of an access code including a referral-ID and a volume-ID.

The volume corresponding information, the referral information, the referral patient information, and the reception information that are illustrated in FIG. 5 to FIG. 8 are only examples, and a part of or all of the items may be omitted or changed in accordance with the use or the conditions of the information processing system. The medical examination information, the analysis target information, and the integrated analysis target information illustrated in FIG. 9 is only an example, and these pieces of information changes in accordance with the medical examination of the patient and the progress state of treatment at each hospital.

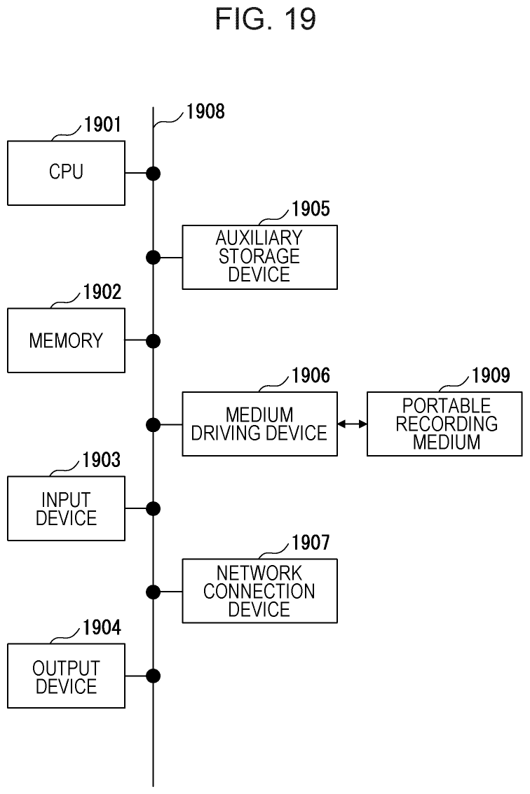

FIG. 19 illustrates an example of the hardware configuration of the information processing apparatus used for the information processing apparatus 112 in FIG. 1 and the referral information control server 323 in FIGS. 3A and 3B. The information processing apparatus in FIG. 19 includes a central processing unit (CPU) 1901, a memory 1902, an input device 1903, an output device 1904, an auxiliary storage device 1905, a medium driving device 1906, and a network connection device 1907. These components are coupled with each other by a bus 1908.

The memory 1902 is a semiconductor memory, for example, a read only memory (ROM), a random access memory (RAM), a flash memory, or the like and stores a program and data to be used for processing. The memory 1902 may be used for the storage unit 344 in FIGS. 3A and 3B.

The CPU 1901 (processor) executes the program, for example, using the memory 1902 to operate as the allocation unit 131 and the control unit 132 in FIG. 1. The CPU 1901 also executes the program using the memory 1902 so as to operate as the allocation unit 342 and the control unit 343 in FIGS. 3A and 3B.

The input device 1903 is, for example, a keyboard, a pointing device, or the like and used for inputting instruction or information by an operator or a user. The output device 1904 is, for example, a display device, a printer, a speaker, or the like and used for outputting a reply to an inquiry of an operator or a user, or a processing result.

The auxiliary storage device 1905 is, for example, a magnetic disk device, an optical disc device, a magneto-optical disk device, a tape device, or the like. The auxiliary storage device 1905 may be a hard disk drive. It is possible for the information processing apparatus to store a program and data in the auxiliary storage device 1905, to load them into the memory 1902, and to use them. The auxiliary storage device 1905 may be used as the storage unit 344 in FIGS. 3A and 3B.

The medium driving device 1906 drives the portable recording medium 1909 and accesses the recording contents. The portable recording medium 1909 is a memory device, a flexible disk, an optical disc, a magneto-optical disk, or the like. The portable recording medium 1909 may be a digital versatile disk (DVD), a compact disk read only memory (CD-ROM), a Universal Serial Bus (USB) memory, or the like. It is possible for an operator or a user to store the program and the data in the portable recording medium 1909, to load them into the memory 1902, and to use them.

In this manner, a computer-readable recording medium that stores a program and data used for processing is a physical (non-transitory) recording medium, such as a memory 1902, an auxiliary storage device 1905, or a portable recording medium 1909.

The network connection device 1907 is a communication interface that is connected to a communication network, such as a local area network (LAN), a wide area network (WAN), or the like and performs data conversion involved in the communication. It is possible for the information processing apparatus to receive a program and data from an external device via a network connection device 1907, to load them into the memory 1902, and to use them.

The information processing apparatus does not have to include all the components in FIG. 19, and it is possible to omit a part of the components in accordance with the use or the conditions. For example, when an instruction or information from an operator or a user does not have to be input, the input device 1903 may be omitted. When a reply to an inquiry from an operator or a user, or processing result does not have to be output, the output device 1904 may be omitted. When a portable recording medium 1909 is not used, the medium driving device 1906 may be omitted.

It is possible to use the same information processing apparatus as that in FIG. 19 for the doctor terminal 301-1, the medical office terminal 302-1, the MMS terminal 303, the doctor terminal 301-2, the medical office terminal 302-2, and the mobile terminal 304 in FIGS. 3A and 3B. It is also possible to use the same information processing apparatus as that in FIG. 19 for the analyst terminal 311, the server 312, the storage device 313, the aggregation device 314, the server 315, and the storage devices 316 to 318. It is also possible to use the same information processing apparatus as that in FIG. 19 for the reception server 319, the issuing device 320, the storage device 321, and the management server 322.

All examples and conditional language provided herein are intended for the pedagogical purposes of aiding the reader in understanding the invention and the concepts contributed by the inventor to further the art, and are not to be construed as limitations to such specifically recited examples and conditions, nor does the organization of such examples in the specification relate to a showing of the superiority and inferiority of the invention. Although one or more embodiments of the present invention have been described in detail, it should be understood that the various changes, substitutions, and alterations could be made hereto without departing from the spirit and scope of the invention.

What is claimed is:

1. An information processing system, comprising:
a storage device including a first memory including a plurality of physical volumes;
a first information processing apparatus including a first processor configured to:
receive referral information of a patient from a first terminal of a first medical institution;
allocate, upon receiving the referral information, a predetermined physical volume to be used for storing medical examination information of the patient, from among the plurality of physical volumes; and
generate volume identification information that indicates the predetermined physical volume; and
a second information processing apparatus including a second processor configured to
store a copy of the medical examination information of the patient in the predetermined physical volume in accordance with a request received from the first information processing apparatus, the medical examination information of the patient being stored in a first database of the first medical institution,
wherein
the first processor is further configured to:
receive the volume identification information from a second terminal of a second medical institution;
identify the predetermined physical volume based on the volume identification information received from the second terminal; and
allocate the identified physical volume to the second processor, and
the second processor is further configured to:
obtain the copy of the medical examination information of the patient from the predetermined physical volume; and
store the obtained copy of the medical examination information in a second database of the second medical institution.

2. The information processing system according to claim 1, wherein
the first processor is further configured to:
issue, upon receiving the referral information, user identification information that identifies the patient to the first medical institution;
store the volume identification information in a second memory included in the first information processing apparatus, in association with the user identification information;
receive the user identification information from a third terminal;
transmit the volume identification information to the third terminal based on the user identification information received from the third terminal.

3. The information processing system according to claim 2, wherein
the referral information includes referral identification information that identifies the referral information, and
the first processor is further configured to:
store the referral information received from the first terminal in the second memory in association with the user identification information;
transmit the referral identification information stored in the second memory to the third terminal based on the user identification information received from the third terminal;
identify the referral information based on the referral identification information received from the second terminal; and
transmit the identified referral information to the second processor, and
the second processor is further configured to
transmit the referral information received from the first processor to the second terminal.

4. The information processing system according to claim 3, wherein
the first processor is further configured to:
identify, based on the referral identification information received from the second terminal, reception information stored in the second memory in association with the referral identification information, the reception information including information indicating a preparatory state for receiving the patient by the second medical institution; and
transmit the identified reception information to the second processor, and
the second processor is further configured to
transmit the reception information received from the first processor to the first terminal.

5. The information processing system according to claim 1, wherein
the second processor is further configured to:
add first identification information indicating the first medical institution to medical examination information generated in the first medical institution to obtain first medical examination information;
store the first medical examination information in the first database;
store a copy of the first medical examination information in the predetermined physical volume;
extract the first medical examination information of the patient from the first database;
generate analysis target information of the first medical institution based on the extracted first medical examination information of the patient in accordance with analysis target item information that specifies an item with which an individual is not identified;
store a copy of the first medical examination information in the second database;
add second identification information indicating the second medical institution to medical examination information generated in the second medical institution to obtain second medical examination information;
store the second medical examination information in the second database;
extract the second medical examination information of the patient from the second database;
generate analysis target information of the second medical institution based on the extracted second medical examination information of the patient in accordance with the analysis target item information, and
the information processing system further comprises
a third information processing apparatus including a third processor configured to
merge the analysis target information of the first medical institution and the analysis target information of the second medical institution to generate integrated analysis target information.

6. An information processing apparatus, comprising:
a processor configured to:
receive referral information of a patient from a first terminal of a first medical institution;
allocate, upon receiving the referral information, a predetermined physical volume to be used for storing medical examination information of the patient, from among a plurality of physical volumes included in a storage device;
generate volume identification information that indicates the predetermined physical volume;
cause an information processing device to store a copy of the medical examination information of the patient in the predetermined physical volume, the medical examination information of the patient being stored in a first database of the first medical institution;
receive the volume identification information from a second terminal of a second medical institution;
identify the predetermined physical volume based on the volume identification information received from the second terminal;
allocate the identified physical volume to the information processing device; and
cause the information processing device to obtain the copy of the medical examination information of the patient from the predetermined physical volume and store the obtained copy of the medical examination information in a second database of the second medical institution.

7. A non-transitory computer-readable recording medium having stored therein a program that causes a computer to execute a process, the process comprising:
receiving referral information of a patient from a first terminal of a first medical institution;
allocating, upon receiving the referral information, a predetermined physical volume to be used for storing medical examination information of the patient, from among a plurality of physical volumes included in a storage device;
generating volume identification information that indicates the predetermined physical volume;
causing an information processing device to store a copy of the medical examination information of the patient in the predetermined physical volume, the medical examination information of the patient being stored in a first database of the first medical institution;
receiving the volume identification information from a second terminal of a second medical institution;
identifying the predetermined physical volume based on the volume identification information received from the second terminal;
allocating the identified physical volume to the information processing device; and
causing the information processing device to obtain the copy of the medical examination information of the patient from the predetermined physical volume and store the obtained copy of the medical examination information in a second database of the second medical institution.
